(19) United States
(12) Patent Application Publication
(10) Pub. No.: US 2018/0028753 A1
(43) Pub. Date: Feb. 1, 2018

(54) High Efficiency Auto-Injector

(71) Applicant: Meridian Medical Technologies, Inc., Columbia, MD (US)

(72) Inventors: John Glyndwr Wilmot, Mount Airy, MD (US); Rajesh B. Shukla, Belle Mead, NJ (US); Clarence Michael Mesa, Boyds, MD (US); Megha V. Mahadevan, Laurel, MD (US)

(21) Appl. No.: 15/484,950

(22) Filed: Apr. 11, 2017

Related U.S. Application Data
(63) Continuation of application No. 14/202,415, filed on Mar. 10, 2014, now abandoned.
Application No. 14/202,415 is a continuation of application No. 12/909,070, filed on Oct. 21, 2010, now abandoned.

(51) Int. Cl. A61M 5/20; A61M 5/32; A61M 5/24; A61M 5/31

(52) U.S. Cl. CPC A61M 5/2033; A61M 5/24; A61M 5/326; A61M 2005/206; A61M 5/3204; A61M 2005/2407; A61M 2005/3125; A61M 5/3271

(57) ABSTRACT

An auto-injector apparatus and associated methods utilizing specific dimensions and parameters of use for the auto-injector are provided for achieving increased effectiveness of the auto-injector device in delivering medicament into the patient's body, and in dispersion of the medicament from the initial injection site into the surrounding bodily tissues.

### FIELD OF THE INVENTION

The invention relates to an automatic injector or auto-injector for delivering medicament to an injection site, and methods of use thereof.

### DESCRIPTION OF THE PRIOR ART

An automatic injector or auto-injector is a device designed to allow a user to self-administer a pre-measured dose of a medicament composition subcutaneously or intramuscularly, usually in an emergency situation. Automatic injectors are used, for example, to treat anaphylactic (severe allergic) reactions and to administer antidotes for certain poisons, such as chemical nerve agents and various drug compositions such as diazepam.

A typical auto-injector has a housing, inside of which is a cartridge. The cartridge has one or several chambers containing medicament compositions or components thereof and is adapted to be attached to a needle assembly. The cartridge can hold either a pre-mixed liquid medicament or a solid medicament and a liquid that are mixed prior to injection. The housing carries an actuation assembly with a stored energy source, for example, a compressed spring. Activation of the actuation assembly causes a sequence of movements, whereby the needle extends from the auto-injector into the user so that the medicament compound is then forced through the needle and into the user. After delivery of the dose of medicament into the injection site, the needle remains in an extended position. If the auto-injector is of the type designed to carry plural components of the medicament composition in separate, sealed compartments, structure may be included that forces the components to mix when the actuation assembly is activated.

There is a need for an auto-injector having a cover that provides protection from the needle both prior to and after operation of the auto-injector. U.S. Pat. No. 5,295,965 to Wilmot et al., U.S. Pat. No. 6,767,336 to Kaplan, and U.S. Pat. No. 7,449,012 have all previously dealt with such needle covers.

### SUMMARY OF THE INVENTION

An auto-injector apparatus and associated methods utilizing specific dimensions and parameters of use for the auto-injector are provided for achieving increased effectiveness of the auto-injector device in delivering medicament into the patient's body, and in dispersion of the medicament from the initial injection site into the surrounding bodily tissues

An auto-injector apparatus in one embodiment includes a housing, a cartridge disposed in the housing and containing a medicament, the medicament rearwardly confined by a plunger, the cartridge including a needle to dispense the medicament there through, the needle having an inside diameter of at least 0.0115 inch. The auto-injector further includes an actuation assembly having a stored energy source capable of being released to drive the plunger within the cartridge to dispense the medicament through the needle. The energy source delivers a dynamic force of at least about 20 pounds to the plunger as the plunger begins moving relative to the cartridge. The auto-injector apparatus further includes a needle cover at least partially received in the housing, the needle cover having an enclosed end surface having an end opening in the enclosed end surface to permit the needle to pass through the end opening during a medicament dispensing operation. The enclosed end surface has a flat planar annular portion surrounding the end opening and arranged to be placed on an injection surface of a user of the auto-injector to transmit an activation force to the actuation assembly when the auto-injector is pressed against the injection surface. The flat planar annular portion of the enclosed end surface has an area of at least about 0.20 square inches.

A method of automatically injecting a medicament into a user may include;

(a) providing an auto-injector apparatus, including:

a housing;

a cartridge contained in the housing, the cartridge containing at least about 0.15 mL of medicament and including a plunger engaging the medicament and a needle connected to the cartridge;

an actuating assembly operably associated with the cartridge and the plunger; and

a needle guard operably associated with the actuating assembly;

(b) placing a flat planar end surface of the needle guard against an injection site of the user, the end surface having a surface area of at least about 0.20 square inches;

(c) pressing the end surface of the needle guard against the injection site with a force of at least about 2 pounds and thereby actuating the actuating assembly of the auto-injector apparatus so that:
(c)(1) the needle extends from the apparatus into the user, the needle having a needle bore diameter of at least 0.0115 inch; and
(c)(2) a force of at least about 20 pounds is applied by the plunger to the medicament so that at least about 0.15 mL of the medicament is expelled through the needle into the user within no more than about 0.5 second;

(d) after step (c), holding the end surface against the injection site for at least about 5 seconds; and

(e) after step (d), removing the end surface from contact with the injection site and automatically extending the end surface to cover the needle.

### BRIEF DESCRIPTION OF THE DRAWINGS

An understanding of the various embodiments of the invention may be gained by virtue of the following figures, of which like elements in various figures will have common reference numbers, and wherein:

FIG. 1 is a side cross sectional view of an embodiment of an auto-injector, including notations of some specific dimensions and parameters of interest, along with an indication of the location within the auto-injector of the components associated with those dimensions and parameters;

FIG. 2 is a side cross sectional view of the auto-injector of FIG. 1 in an unactivated state having the release pin in place;

FIG. 3 is a side schematic view of the auto-injector in the unactivated state of FIG. 2;

FIG. 4 is a side cross sectional view of the auto-injector of FIG. 1 having the release pin removed in preparation for activation;

FIG. 5 is a side cross sectional view of the auto-injector of FIG. 1 wherein the needle cover spring is in a compressed state;

FIG. 6 is a side schematic view of the auto-injector of FIG. 5;

FIG. 7 is a side cross sectional view of the auto-injector in an actuated state with the needle in a drug delivery position;

FIG. 8 is a side schematic view of the auto-injector of FIG. 7;

FIG. 9 is a side cross sectional view of the auto-injector following delivery of the drug wherein the needle cover is in an extended protective state;

FIG. 10 is an enlarged view of the locking wings of the cartridge container when the needle cover is in the extended protective state, as shown in FIGS. 9 and 11;

FIG. 11 is a side schematic view of the auto-injector of FIG. 9;

FIG. 12 is a left front schematic view of the auto-injector of FIG. 1 having the outer body removed wherein the needle cover is located in a retracted position prior to activation of the auto-injector;

FIG. 13 is an enlarged view of FIG. 12 illustrating the position of the locking wings of the cartridge container and the locking teeth;

FIG. 14 is a left front schematic view of the auto-injector of FIG. 1 having the outer body removed when the needle cover is located in an extended protective position after use of the auto-injector;

FIG. 15 is an enlarged view of FIG. 14 illustrating the position of the locking wings of the cartridge container and the locking teeth;

FIG. 16 is an enlarged cross sectional view illustrating the position of the locking teeth when the needle cover is in the extended protective position;

FIG. 17 is a left rear perspective view of the power pack outer body for the power pack for the auto-injector;

FIG. 18 is a side perspective view of the collet for the power pack for the auto-injector;

FIG. 19 is a right front perspective view of the power pack inner body for the power pack for the auto-injector;

FIG. 20 is a side perspective view of the spring assembly for the power pack for the auto-injector;

FIG. 21 is a left bottom perspective view of the release pin for the auto-injector;

FIG. 22 is a right bottom perspective view of the power pack of the auto-injector in an assembled state;

FIG. 23 is a side cross sectional view of the power pack of FIG. 22;

FIG. 24 is a top left perspective view of the power pack of FIG. 22 having the top portion of the release pin and a peripheral rib of the power pack outer body removed;

FIG. 25 is a top left perspective view of the power pack of FIG. 22;

FIG. 26 is a top left perspective view of the power pack positioned within the outer body having the safe pin removed;

FIG. 27 is left side perspective view of the power pack outer body;

FIG. 28 is a partial cross sectional perspective view illustrating the interior of the power pack outer body;

FIG. 29 is a partial cross sectional perspective view illustrating the interior of the power pack inner body;

FIG. 30 is side perspective view of the power pack inner body;

FIG. 31 is a bottom perspective view of the power pack inner body;

FIG. 32 is a side view of the release pin;

FIG. 33 is another side view of the release pin of FIG. 32 rotated 90 degrees about an axis;

FIG. 34 is a bottom perspective view of the safe pin of FIG. 32;

FIG. 35 is a side view of the collet of the power pack;

FIG. 36 is another side view of the collet of FIG. 35 rotated 90 degrees about an axis;

FIG. 37 is an enlarged end view of the collet illustrating the stabilizing arch;

FIG. 38 is side perspective view of the needle cover located within the outer body of the auto-injector;

FIG. 39 is a cross sectional view of the cartridge container and needle cover located within the outer body with the power pack removed prior to final assembly of the auto-injector;

FIG. 40 is a cross sectional view of the cartridge container and needle cover located within the outer body of FIG. 39 rotated 90 degrees about an axis with the power pack removed prior to final assembly of the auto-injector;

FIG. 41 is a front left side perspective view of the cartridge container of the auto-injector;

FIG. 42 is a perspective view of the needle cover spring;

FIG. 43 is a front left side perspective view of the needle cover of the auto-injector;

FIG. 44 is a front left side perspective view of the outer body of the auto-injector;

FIG. 45 is another left side perspective view of the outer body of FIG. 44;

FIG. 46 is a partial cross sectional perspective view illustrating the interior of the outer body;

FIG. 47 is a side view of the outer body;

FIG. 48 is another side view of the outer body of FIG. 47 rotated 90 degrees about an axis;

FIG. 49 is a right rear side perspective view of the cartridge container of the auto-injector;

FIG. 50 is a side view of the cartridge container;

FIG. 51 is another side view of the cartridge container of FIG. 51 rotated 90 degrees about an axis;

FIG. 52 is an enlarged side view of the cartridge container illustrated in FIG. 51, wherein the dotted lines illustrate the deflection path of the locking wings;

FIG. 53 is a right rear perspective view of the needle cover of the auto-injector;

FIG. 54 is a side view of the needle cover of FIG. 53;

FIG. 55 is a perspective view of the needle cover spring;

FIG. 56 is a right top perspective view of a locking tooth of the auto-injector;

FIG. 57 is a left bottom perspective view of the locking tooth of FIG. 55;

FIG. 58 is a side view of the locking tooth;

FIG. 59 is a top view of the locking tooth;

FIG. 60A is a line drawing reproduction of a CT scan visualizing injectate volumes at 1 minute on the left and 15 minutes on the right for EpiPen® vs. Anapen® 300.

FIG. 60B is a line drawing reproduction of a CT scan visualizing injectate volumes at 1 minute on the left and 15 minutes on the right for EpiPen® Jr. vs. Twinject® 0.15 mL;

FIG. 60C is a line drawing reproduction of a CT scan visualizing injectate volumes at 1 minute on the left and 15 minutes on the right for EpiPen® vs. Twinject® 0.30 mL;

FIG. 61 is a graph comparing the average efficiency of uptake of delivered injectate into the muscle tissue of each of the groups of test pigs for both the test articles and the control articles;

FIG. 62 is a line drawing reproduction of an initial tomogram (scout) image;

FIG. 63 is a line drawing reproduction of a screenshot of the software used in the testing;

FIG. 64 is a graph comparing the efficiency of uptake of delivered injectate into the muscle tissue of one test pig for Anapen® 300 vs. EpiPen® injectors;

FIG. 65 is a graph comparing the efficiency of uptake of delivered injectate into the muscle tissue of one test pig for Anapen® 300 vs. EpiPen® injectors;

FIG. 66 is a graph comparing the efficiency of uptake of delivered injectate into the muscle tissue of one test pig for Twinject® 0.15 mL vs. EpiPen® Jr. injectors;

FIG. 67 is a graph comparing the efficiency of uptake of delivered injectate into the muscle tissue of one test pig for Twinject® 0.15 mL vs. EpiPen® Jr. injectors;

FIG. 68 is a graph comparing the efficiency of uptake of delivered injectate into the muscle tissue of one test pig for Twinject® 0.30 mL vs. EpiPen® injectors;

FIG. 69 is a graph comparing the average efficiency of uptake of delivered injectate into the muscle tissue for the group P3 test pigs for Twinject® 0.30 mL vs. EpiPen® injectors;

FIG. 70 is a graph comparing the average efficiency of uptake of delivered injectate into the muscle tissue for the three test articles: Twinject® 0.30 mL; Twinject® 0.15 mL; and Anapen® 300 injectors;

FIG. 71 is a graph comparing the average efficiency of uptake of delivered injectate into the muscle tissue for the three control articles: EpiPen® (P1 tests); EpiPen® Jr. (P2 tests); and EpiPen® (P3 tests);

FIG. 72 is a graph comparing the average efficiency of uptake of delivered injectate into the muscle tissue of the group P1 test pigs for denim patch vs. direct skin auto-injectors;

FIG. 73 is a graph comparing the average efficiency of uptake of delivered injectate into the muscle tissue of the group P2 test pigs for denim patch vs. direct skin auto-injectors;

FIG. 74 is a graph comparing the average efficiency of uptake of delivered injectate into the muscle tissue of the group P3 test pigs for denim patch vs. direct skin auto-injectors;

FIG. 75 is a schematic illustration of the injection site on a user, showing the compression of the body tissue around the needle.

### DETAILED DESCRIPTION OF THE INVENTION

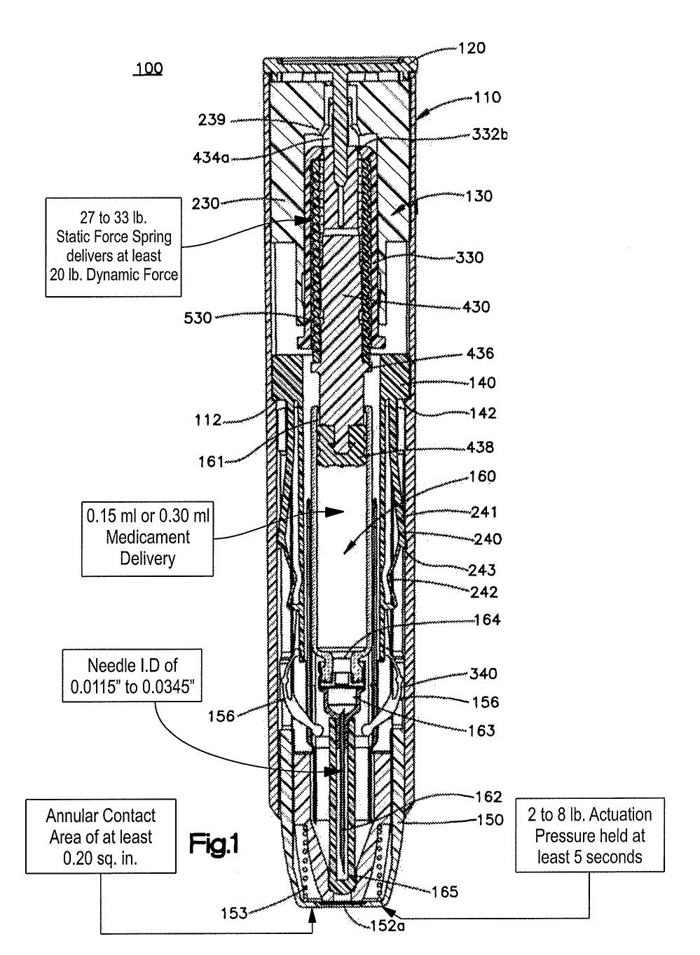

An auto-injector apparatus and associated methods of use are disclosed. The apparatus and methods utilize specific dimensions and parameters of use to provide increased effectiveness of the auto-injector device in delivering medicament into the patient's body, and in dispersion of the medicament from an initial injection site into the surrounding bodily tissues. In FIG. 1 the auto-injector 100 is shown with notations of some of the specific dimensions and parameters of interest, along with an indication of the location within the auto-injector 100 of the components associated with those dimensions and parameters.

It should be appreciated that some of the components described herein are conventionally known in the broader aspects, as described in U.S. Pat. No. 4,031,893 (“the '893 patent”) hereby incorporated by reference in its entirety, and thus not described in unnecessary detail here. It should also be appreciated that known modifications or variations to the '893 patent can apply equally to the auto-injector of the present invention as will be described below. These modifications or variations include embodiments described in U.S. Pat. Nos. 4,226,235; 4,329,988; 4,394,863; 4,723,937; and U.S. Ser. Nos. 09/985,466; 10/285,692, each of which is incorporated by reference in its entirety for the full teachings therein.

The auto-injector 100 includes an outer body or housing 110, a release pin 120, a power pack 130, a cartridge container 140, a needle cover 150 and a cartridge 160 housing a dose of medicament. The dose can be stored in liquid or solid form or as a combination of a liquid and a solid that is mixed prior to injection. The dose can also be stored in the form of two liquids that are mixed prior to injection.

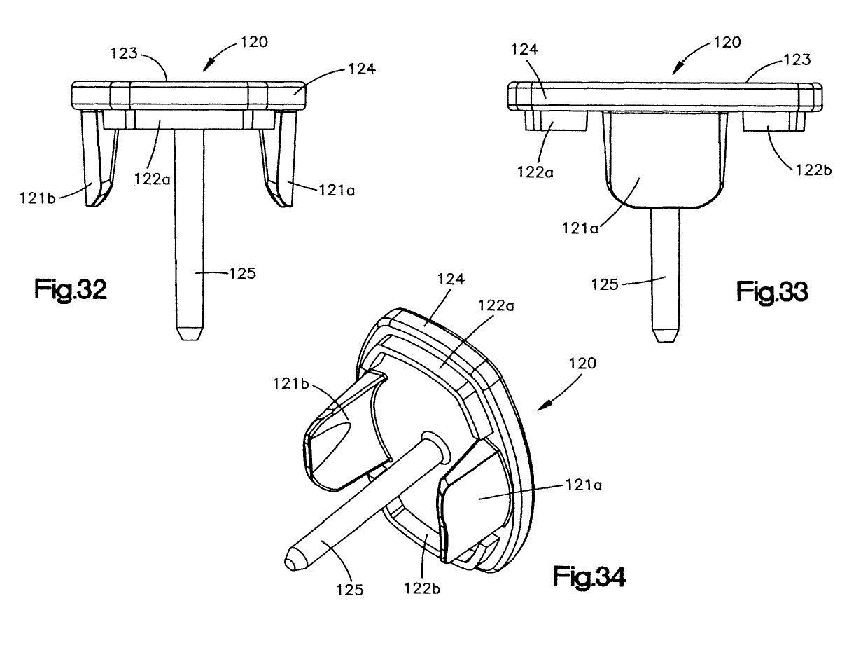

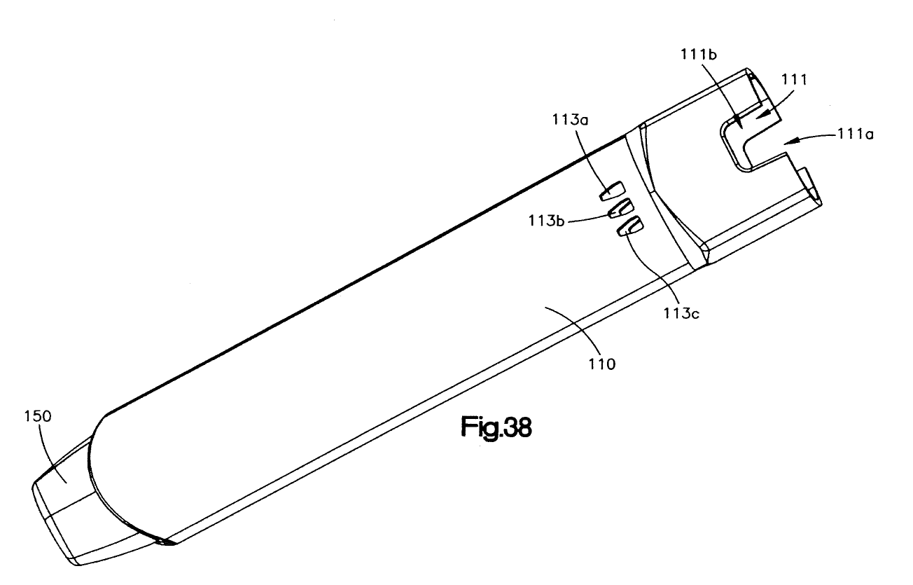

The outer body or housing 110 is shown in FIGS. 38 and 44-48. The outer body 110 has a generally oval or elliptical shape, which is more ergonomic sized to permit easy grasping and use by the user or caregiver in comparison with a cylindrical body. The generally oval shape of the outer body 110 prevents the auto-injector 100 from inadvertently rolling or sliding off a flat surface. Furthermore, the oval shape provides a larger print surface for labeling the auto-injector 100 with instructions. The outer body 110 is preferably formed from a synthetic material such that it can be easily molded. The outer body 110 can be transparent such that the interior components can be easily viewed through the outer body 110. With such a construction, the user can view the contents of the cartridge 160 through windows 141a and 141b in the cartridge container 140 and the needle cover 150 at predetermined times. It is also contemplated that the outer body 110 can be opaque such that the interior components are not visible through the outer body 110. It is also contemplated that the outer body 110 has a window or windows that permit viewing of the components within the outer body 110. The outer body 110 has an opening 111 formed in one end that is sized to receive a release pin 120. When in place, the release pin 120 prevents inadvertent use or activation of the auto-injector 100. The release pin 120 is illustrated in FIGS. 32-34. It is contemplated that operating instructions may be printed directly onto the outer body 110. It is also contemplated that a label may be affixed to the outer body 110, which may increase the rigidity of the outer body 110. When the outer body 110 includes one or more apertures, the provision of a label increases the strength of the outer body 110, which makes the provision of additional structural reinforcements unnecessary.

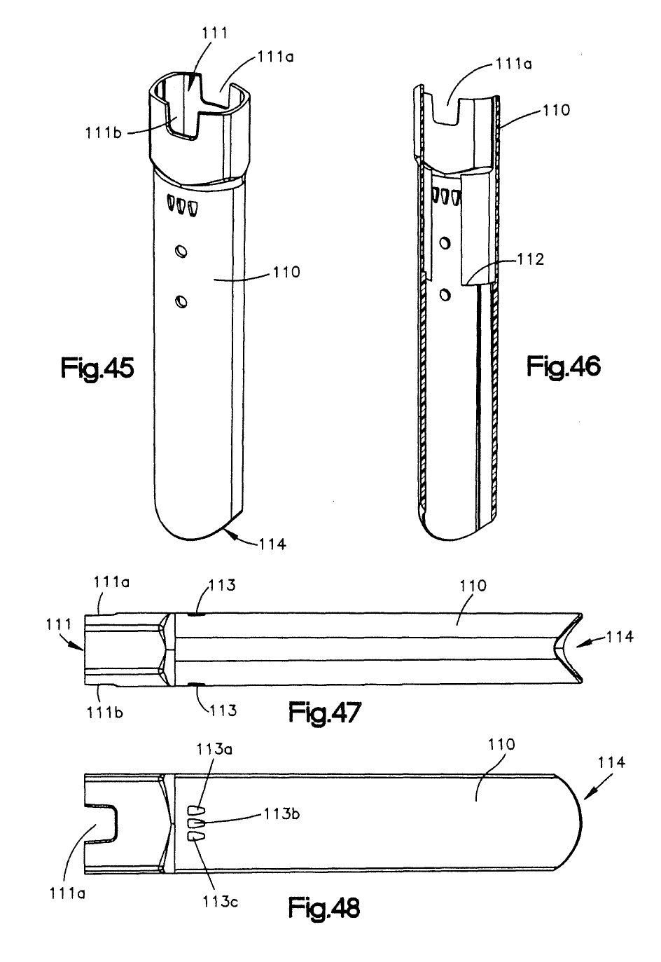

The opening 111 includes side recesses 111a and 111b, which extend downwardly along opposing sides of the outer body 110, shown in FIGS. 45, 46 and 48. While two recesses are shown, it is contemplated that a single recess may be provided or more than two may be provided. The number of recesses will correspond to the number of tabs. The recesses 111a and 111b are sized so that they may receive downwardly extending tabs 121a and 121b on the release pin 120. The tabs 121a and 121b prevent rotation of the release pin 120 such that the user easily recognizes that the release pin 120 is to be pulled rather than rotated to permit removal of the release pin 120 in order to actuate the auto-injector 100. The tabs 121a and 121b are primarily received in retention recesses 235 located on opposing sides of the power pack 130, described in greater detail below. The recesses 111a and 111b provide access to the tabs 121 in the recesses 235. The tabs 121a and 121b are compression fit onto the power pack 130 to prevent inadvertent removal. To release the pin 120, the operator compresses or pinches the tabs 121 to dislodge the edges of the tabs 121 from the recesses 235 such that the pin 120 can then be pulled/removed from the power pack 130. As shown, the tabs 121 have a curvature which creates a chamfered edge that engages the edges of the recesses 235. The shape of the tabs 121 and the recesses 235 are full complimentary, which creates the friction or compressive retaining force between the pin 120 and power pack 130. The release pin 120 also includes downwardly projecting ribs 122a and 122b, which are adapted to be received on the top surface of the power pack 130. The ribs 122a and 122b increase the stability and rigidity of the release pin 120. It is contemplated that additional ribs may be provided. The release pin 120 includes an outwardly facing flat end 123 having a peripheral ledge 124. The peripheral ledge 124 permits grasping of the release pin 120 by the user. The ledge 124 is sized to rest on the end surface of the outer body 110 adjacent opening 111. The release pin 120 includes a downwardly extending pin 125, which engages the collet 430 of the power pack 130. When secured in place (i.e., prior to removal of the release pin 120 and prior to actuation of the auto-injector 100), the pin 125 prevents the end of the collet 430 from compressing, which prevents actuation of the auto-injector 100. The end 123 has a shape corresponding to the oval/elliptical shape of the outer body 110.

As shown in FIG. 46, the inner surface of the outer body 110 is contoured to receive the power pack 130, a cartridge container 140 and a needle cover therein 150. Unlike many prior art needle covers, the needle cover 150 is positioned between the container 140 and the outer body 110 such that the user does not contact the cover 150 during the operation, which could impede the deployment of the cover or cause a diaphragm within the cartridge to rupture prematurely. Additionally, the mechanisms for locking and deploying the cover member are located within the outer body 110 and are thus protected against tampering and dirt ingress. The outer body 110 includes a cartridge container retention step 112 formed on the inner surface near the end of the outer body 110 adjacent the opening 111. A ledge 142 of the cartridge container 140 abuts the retention step 112 to limit the downward movement of the cartridge container 140 within the outer body 110 once the auto-injector 100 has been assembled such that the container cannot be moved out of opening 114. A plurality of power pack retention openings 113a, 113b and 113c are formed on at least one side of the outer body 110. Projections or teeth 238 on the power pack 130 are snap fit into the openings 113. This snap fit prevents the removal of the power pack 130 from the outer body 110 once installed in the outer body 110. The power pack outer body 230 is not movable with respect to the outer body 110. The ledge 142 of the cartridge container 140 is sandwiched between the retention step 112 and the power pack 130.

An opening 114 is formed in the outer body 110 on an end opposite the opening 111. The opening 114 is configured such that a portion of the cartridge container 140, a portion of the needle cover 150 can extend therefrom. The step 112 limits the travel of the container 140 through opening 114. The end of the outer body 110 is intended to be orientated adjacent the injection surface of the user such that end portion of the cover 100 contacts the injection surface.

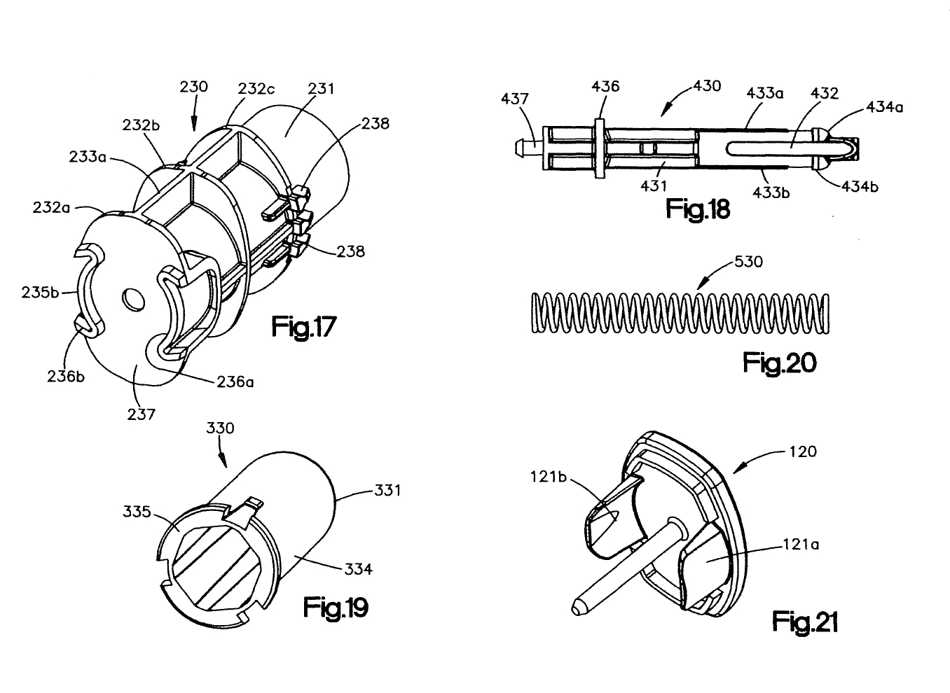

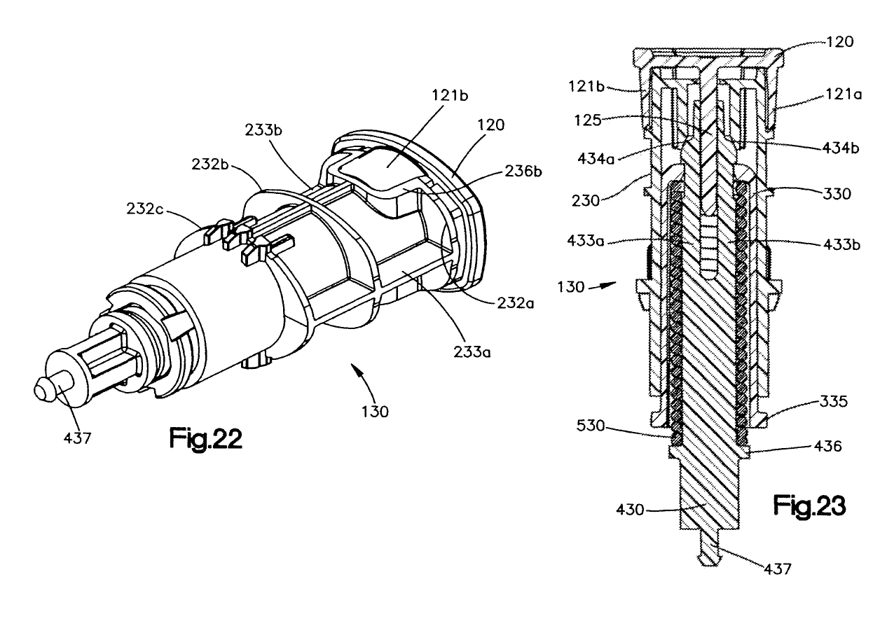

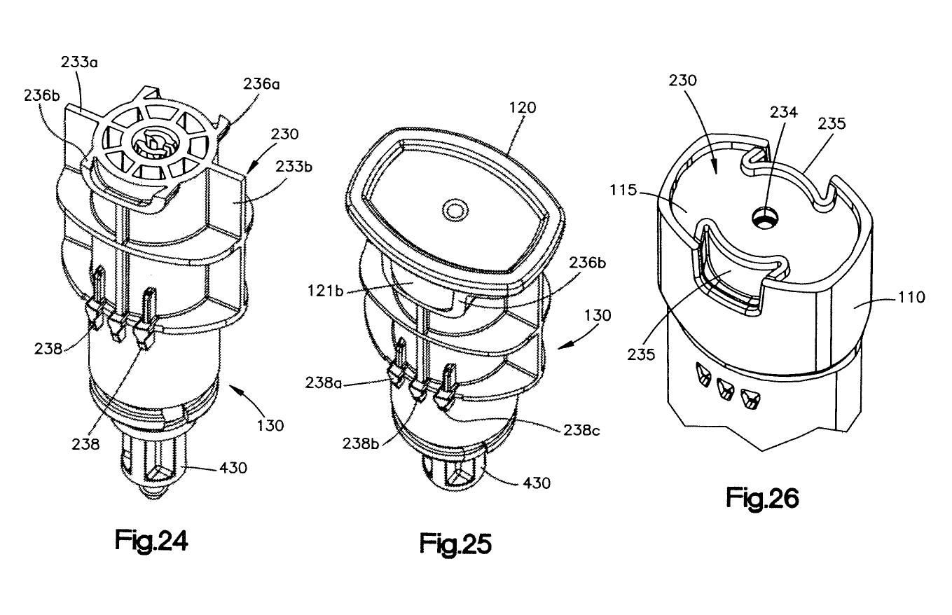

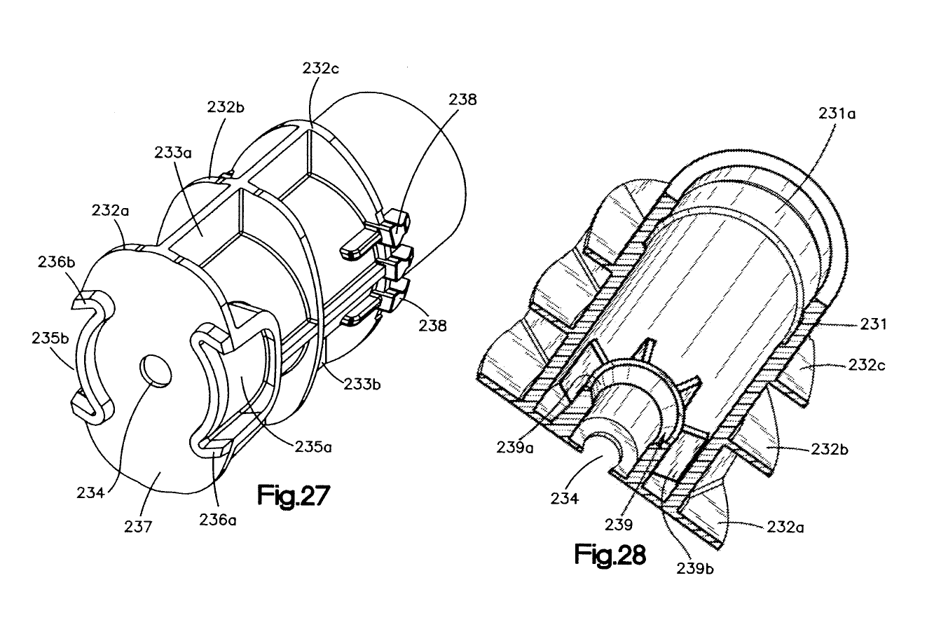

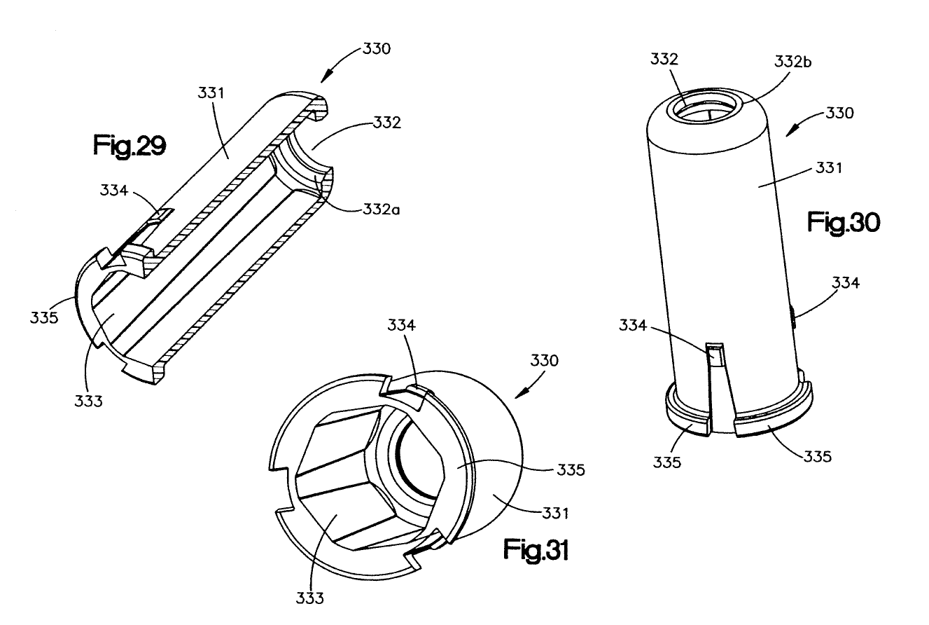

The power pack 130 will now be described in greater detail in connection with FIGS. 17-20, 22-31 and 35-37. The power pack 130 includes a power pack outer body 230, a power pack inner body 330, a collet 430, and a power pack spring assembly 530. The activation force necessary to release the energy stored in the power pack is between 4 to 8 pounds. The activation force is the force required to release the collet 430 from the inner body 330 when the auto-injector 100 is pressed against the injection surface. The injection force provided by the spring assembly 530 is approximately 30 pounds. The injection force must be sufficient such that the cartridge 160 is advanced within the cartridge container 140 to drive the needle such that it pierces the sheath to permit injection of the medicament into the user. The power pack outer body 230 is a generally cylindrical elongated hollow body 231. A plurality of outer peripheral ribs 232a, 232b and 232c extend outwardly from an outer surface of the hollow body 231. While these ribs 232 are shown, it is contemplated additional ribs may be provided. The ribs 232 are provided to prevent distortion of the outer body 110 of the auto-injector 100. A plurality of outer longitudinal ribs 233a, 233b are spaced about the outer surface of the hollow body 231. The ribs 233 cooperate with the ribs 232 to further strengthen the auto-injector 100 and prevent distortion of the outer body 110 when gripped and used by a user.

One of the peripheral ribs 232a forms a top end surface 237 of the power pack outer body 230. A hole 234 is provided in end surface which is sized to receive the downwardly extending pin 125 of the release pin 120. Retention recesses 235a and 235b are formed on opposing sides of the hollow body 231 adjacent the top end surface. The recesses 235a and 235b are formed by walls 236a and 236b which extend outwardly from the hollow body 231 and upwardly from the top end surface 237 of the peripheral rib 232a. The recesses 235a and 235b are aligned with the side recesses 111a and 111b of the outer body 110 such that when the release pin 120 is secured to the auto-injector 100, the tabs 121a and 121b are received in both recesses 235a and 235b. The recesses 235a and 235b are sized to apply a compressive force on the tabs 121a and 121b to secure the release pin 120 in place to prevent inadvertent removal.

As shown in FIGS. 17, 26 and 27, the walls 236a and 236b extend upwardly from the end surface 237 of the peripheral rib 232a. With such an arrangement, the end surface 237 is spaced or recessed below the end surface of the outer body 110, as shown in FIG. 26, forming a recess 115. The recess 115 reduces and/or avoids the visual effect of a push button. As such, the user will not be inclined to press the end surface 237 to administer the medicament. Additionally, it provides a visual indication to the user that the recess 115 is located at the inoperative end of the auto-injector 100 such that the user is inclined to place the cover 150 against the injector surface not the opposite end of the auto-injector. The recess 115 also serves to space the hole 234 from the end of the auto-injector 100 to deemphasize the presence of the hole 234 such that it is hidden when the user reads the label on the outer body 110. As such, the user is disinclined to position the hole 234 adjacent the injection site. This arrangement is just one countermeasure provided to insure against improper use of the auto-injector 100. The ribs 122a and 122b of the release pin 120 are received within the recess 115.

A plurality of projections or teeth 238a, 238b, 238c are formed on the outer surface of the hollow body 231. The teeth 238a, 238b, 238c are sized to be snap fit into the openings 113a, 113b, 113c to secure the power pack 130 within the outer body 110. This construction permits these components 110 and 130 to be secured together without the need of an adhesive of other form of bonding. A corresponding set of teeth 238 may be provided on the opposite side of the hollow body 230 to match the corresponding openings in the outer body 110.

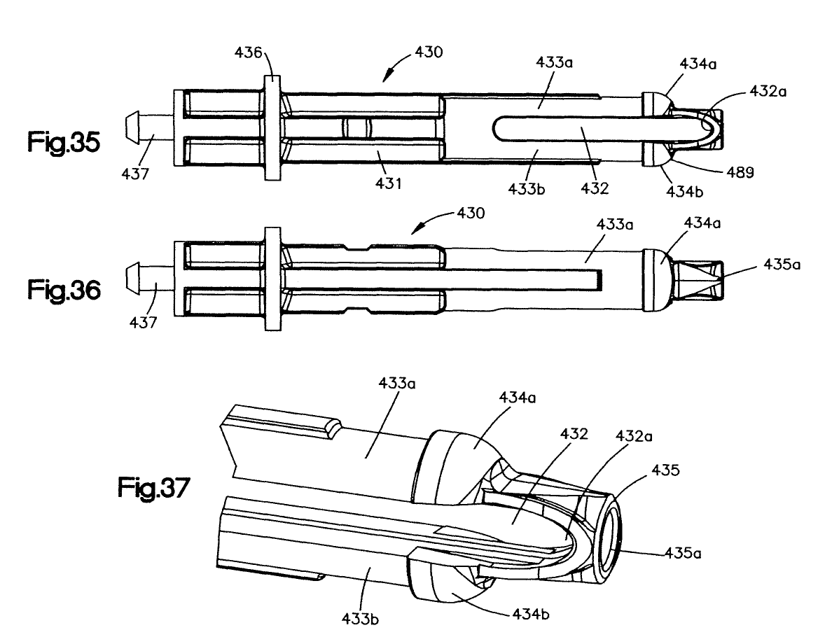

The interior of the hollow body 231 includes a recess 231a, which is sized to receive a retention tab 334 on the power pack inner body 330. The recess 231a may be a groove, which extends about the inner periphery of the hollow body 231. The recess 231a is positioned in the hollow body 231 near an end opposite the end surface 237. As seen in FIGS. 1 and 28, a collet activation structure 239 extends into the interior of the hollow body 231 from the inner side of the end surface 237. The collet activation structure 239 has a generally cylindrical shape with a sloped collet activation surface 239a located on a free end. The activation surface 239a is provided such that when the pin 120 is removed and the front end of the injector is forced into an injection site so that cartridge container 140 rearwardly moves to engage inner body 330, this will rearwardly force the arrowheads 434 and particularly rearward surface 489 thereof (see FIG. 35) into engagement with surface 239a to force the arrowheads 434 of the collet 430 together to release the spring assembly 530 and thus release the necessary energy to inject the medicament into the user. Ribs 239b may be provided to reinforce the collet activation structure 239. It is contemplated that other means of releasing the collet 430 may be employed. A push button type actuation arrangement may be employed, which is described in greater detail in U.S. Pat. No. 4,031,893 and hereby incorporated in its entirety by reference.

The power pack inner body 330 is a generally cylindrical hollow inner body 331. The hollow inner body 331 has an opening 332 formed in one end. The opening 332 has a collet assembly lead-in surface 332a which is used to compress a portion of the collet assembly 430 during assembly of the auto-injector 100 such that is can be properly mounted within the power pack inner body 330. The opening 332 also has a collet retention surface 332b located on an opposite edge which support the opposing arrowheads 434 of the collet 430 prior to activation. The hollow inner body 331 has an opening 333 formed on an opposing end. Spaced from the opening 333 are a plurality of retention tabs 334 which are sized to be snapped into the retention recess 231a. The recess 231 and tabs 334 permit limited movement between the power pack inner body 330 and the power pack outer body 230. The arrangement is also beneficial for purposes of assembling the auto-injector 100. The inner body 330 and the outer body 230 can be preassembled. The recess 231 and tabs 334 maintain the inner body 330 and the outer body 230 in proper alignment for assembly. Furthermore, this arrangement prevents the subassembly of the inner body 330 and the outer body 230 from separating prior to the final assembly in the auto-injector 100. It is also contemplated that other means which permit limited movement between the outer power pack and the inner power pack, which secure the components together may be employed. A ledge 335 at least partially extends about the periphery of the opening 333. The ledge 335 is sized to engage the cartridge container 140 and the power pack outer body 230 at certain times during the operation of the auto-injector 100, described in greater detail below. A spacing exists between the inner power pack 330 and the cartridge container 140 after assembly and prior to activation of the auto-injector 100 to create a gap, which avoids permanently putting forces on the power pack and the spring 530.

A collet 430 is received within the hollow interior of the power pack inner body 330. The collet 430 preferably is a molded one piece construction. The collect 430 has an elongated body 431 having an opening 432 formed therein which forms a pair of side arms 433a and 433b. Each side arm 433a and 433b includes an arrowhead detail 434a and 434b respectively. One side of each arrowhead 434a and 434b is configured to contact and engage the collet retention surface 332b. An opposite side of each arrowhead 434a and 434b is configured to engage the collet assembly lead-in surface 332a, which permits the side arms 433a and 433b to be deflected inwardly to permit operation of the auto-injector 100. The end 435 of the collet 430 adjacent the arrowheads 434a and 434b includes an opening 435a sized to receive the pin 125 of the release pin 120. The pin 125 prevents the side arms 433 from being deflected inwardly towards each other. When secured in place, the pin 125 prevents activation of the auto-injector 100. The opening 432 has an arch 432a formed on one end, as shown in FIG. 37. The arch 432a helps stabilize the side arms 433 and assist them in springing apart when the arms have been compressed together. The arch 432a reduces the amount of stress on the collet.

The collet 430 is positioned within the power pack spring assembly 530. One end of the spring assembly 530 is supported on a flange 436 formed on the collet 430. The flange 436 extends outwardly from the elongated body 431. While the flange 436 supports one end of the spring assembly 530, the location of the flange 436 on the body 431 can also serve to define the delivered dose volume of medicament injected into the user. In certain applications it is desirable to control the amount of medicament delivered through the needle such that a portion of the medicament remains in cartridge 160. The flange 436 may limit the distance that the collet 430 can travel into the cartridge 160, which contains the liquid medicament. As such, the amount of medicament delivered is controlled. In this arrangement, the flange 436 is sized to contact the end of the cartridge 160. For larger diameter cartridges and for larger doses of medicament, it is contemplated that the flange 436 can travel within the cartridge 160. The collet 430 further includes a projection 437, which receives a plunger 438. The plunger 438 is slidably received within the cartridge 160. In other applications, it is desirable to dispense all of the medicament from the container 160. A small residual amount of medicament remains in the needle 162 and the neck of the cartridge 160 adjacent the needle 1 62. In these applications, the flange 436 travels within the interior of the cartridge 160 so that the plunger 438 travels the length of the interior of the cartridge 160 to dispense all of the medicament (except for the residual amounts mentioned above) through the needle 162. It is contemplated that different sized collets 430 may be used in the present auto-injector 100. As such, the collet 430 can be changed based upon cartridge size and desired dose.

The collet 430 is preferably formed as a single piece from a suitable plastic material. The one piece collet 430 simplifies manufacturing and lowers costs by reducing the number of components needed to form a collet. In conventional collets, multiple brass components may be used. In addition in other auto-injectors, a spacer has been required for use in conjunction with the collet 430 to accommodate different amounts of medicament for different auto-injectors. The collet 430 eliminates the multi component construction and also advantageously eliminates the need for a spacer. The length of the collet can be selected based upon the desired dosage. This construction further permits the elimination of a metal insert typically found in the plunger and a firing seat above the power pack inner body. It is contemplated that the size and shape of the collet 430 itself may be varied to accommodate different sized cartridges 160. When the flange 436 does not contact the cartridge 160, it is possible to dispense the entire contents of the cartridge 160 except for any residual amounts remaining in the needle or in the neck of the cartridge 160. It is contemplated that a nipple plunger, as disclosed in U.S. Pat. No. 5,713,866 to Wilmot, the disclosure of which is hereby incorporated specifically herein by reference, may be employed to prevent any buildup of residual amounts of medicament in the neck of the cartridge 160. The position of the flange 436 can be varied to control the amount of dosage injected into the user when the flange is positioned such that the collet and the plunger 438 travel a greater distance within the cartridge 160 before the flange 436 contacts the cartridge 160, a larger dose is dispensed. The length of the collet 430 and the diameter of the cartridge 160 can be selected to control the flow of fluid through the needle 162 of the cartridge 160 so that a desired flow rate is obtained. The auto-injector 100 is configured such that collets 430 of varying sizes can be used within the same outer body 110 and the power pack 430.

An opposite end of the spring assembly 530 rests against an inner surface of the power pack inner body 330 against opening 332.

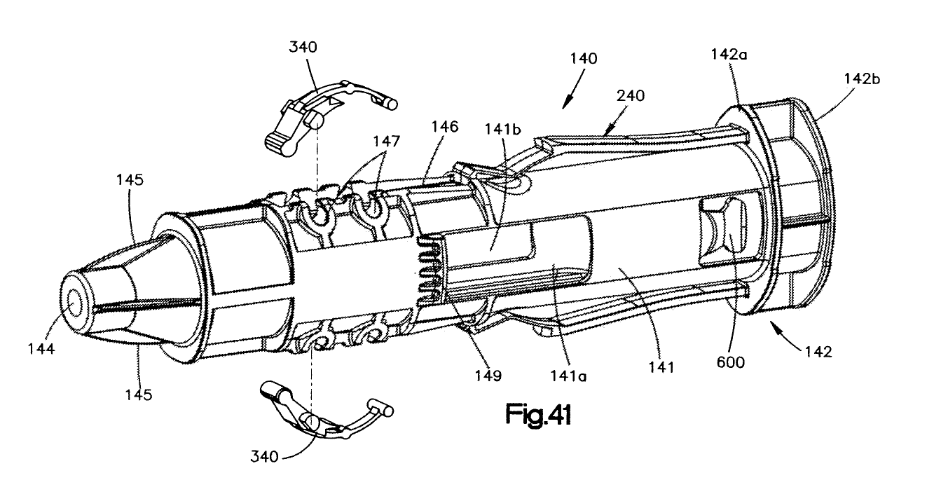

The cartridge container 140 will now be described in greater detail in connection with FIGS. 41 and 49-52. The cartridge container 140 has a generally elongated hollow body 141 sized to be received within the outer body 110. A ledge 142 is formed on one end of the elongated body 141. The ledge 142 contacts the retention step 112 formed on the inner surface of the outer body 110. The ledge 142 limits the downward movement of the cartridge container 140 within the outer body 110 such that it cannot be removed through opening 114. The ledge 142 is formed by peripheral ribs 142a and 142b, which extend outwardly similar to the ribs 232a, 232b and 232c on the power pack outer body 230. The ribs 142a and 142b also prevent distortion of the outer body 110.

The elongated hollow body 141 has a hollow interior sized to receive the cartridge 160 therein. The hollow body has an opening 143 such that the cartridge 160 can be located in the hollow interior and to permit the collet 430 to be slidably received within the cartridge 160. The cartridge container 140 and the locking teeth 340 thereof are designed to accommodate various sized cartridges 160, while maintaining full needle cover functionality. As such, a common design needle cover assembly (including the cartridge container and locking teeth) can be used for various different volumes of drugs and different sized needles. For longer and larger cartridges, it is desirable to provide additional support to prevent axial and radial movement, which could damage or fracture the cartridge 160. A pair of tabs 600 are formed on the hollow body 141 to apply a compressive force on the cartridge 160 to hold and align the cartridge 160 in a proper orientation to prevent such axial and radial movement. The tabs 600 provide friction to prevent movement of the cartridge 160 within the hollow body 141 during shock loading to prevent the cartridge from being dislodged or moved forward with the cartridge holder 140 prior to the medicament dispensing sequence. Typically, the smaller cartridges do not contact the tabs 600. The collet 430 and the needle and needle sheath provide sufficient support for the cartridge. The end of hollow body 141 has a tapered construction with an opening 144 sized to permit the passage there through of the needle 162 and protective sheath 165 of the cartridge 160. A plurality of ribs 145 are formed on the outer surface of the hollow body 141 on the tapered end. The ribs 145 help stabilize the needle cover spring 153 of the needle cover 150. The ribs 145 also serve as guides to aid in the assembly of the auto-injector 100.

The elongated hollow body 141 has at least one viewing window 141a and 141b formed therein. The viewing windows 141a and 141b permit the user to view the contents of the cartridge 160 before activation of the auto-injector 100 to insure that the medicament has not become contaminated or expired.

A pair of locking arms or wings 240 extend from the ledge 142 and are connected to a mid-portion of the hollow body 141, as shown in FIG. 52. Each locking wing 240 has a thickened strut 241 having a generally curved shape, as shown in FIG. 52. The thickened strut 241 is curved such that when a compressive load is applied to the locking wing 240 (e.g., when a user is attempted to push the needle cover 150 back into the outer body 110 after use of the auto-injector 100) the thickened strut 241 bends in the manner illustrated by the dashed lines in FIG. 52. With such a construction, the locking wings 240 are supported by the body 141 of the cartridge container 140, which increases the compressive strength of the locking wings 240. While not preferred, it is contemplated that a single locking wing 240 can be provided.

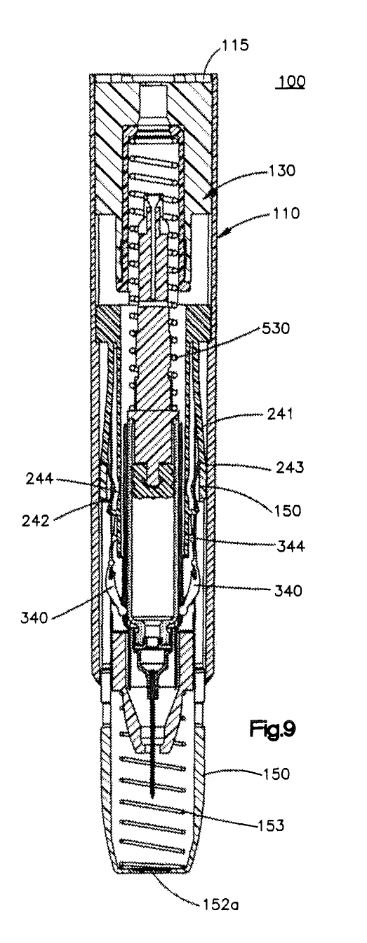

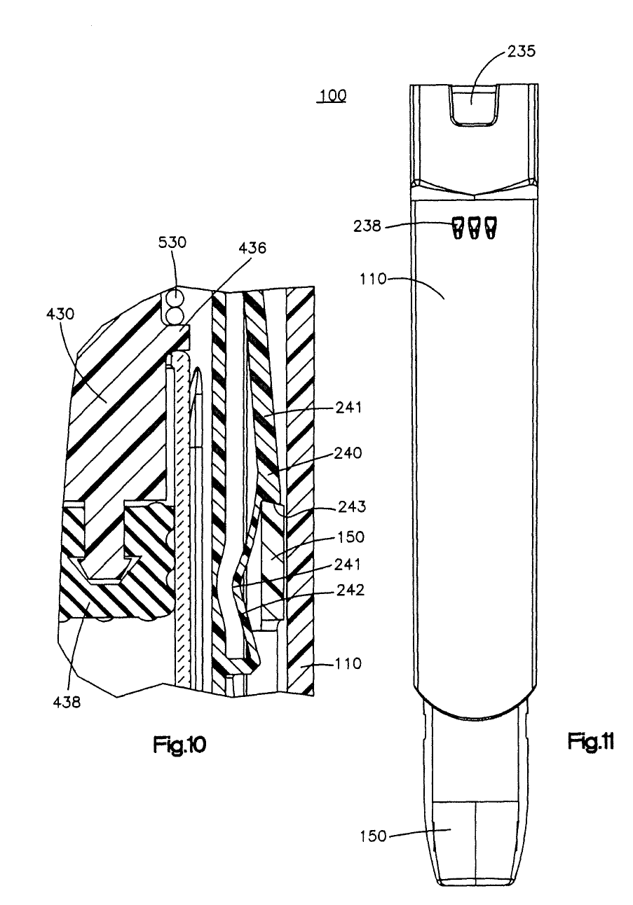

A thinner strut 242 extends from the free end of the strut 241 and is connected to the body 141 of the cartridge container 140. A locking surface 243 is formed at the intersection of struts 241 and 242. The locking surface 243 engages a surface on the cover 150 to limit the inward travel of the cover 150 after operation of the auto-injector 100, as shown in FIGS. 9 and 10. The thinner strut 242 provides a spring force to keep the thicker strut 241 biased in an outwardly direction. The thinner strut 242 also provides tensile strength under extreme loads and helps prevent the strut 241 from collapsing in a sideways direction because the thinner strut 242 remained retained in a guide groove in the needle cover 150 after the cover member 150 has moved to an extended position. The curved shape of the strut 242 permits the strut 242 to bend inwardly as shown in the dashed lines in FIG. 52. This prevents the entire wing 240 from forming a rigid arch. Thus allowing the thicker strut 241 to flex inwardly towards the body 141 without causing excessive compressive leads along the wing 240. It is contemplated that the locking arm 240 may be located on the outer body 110.

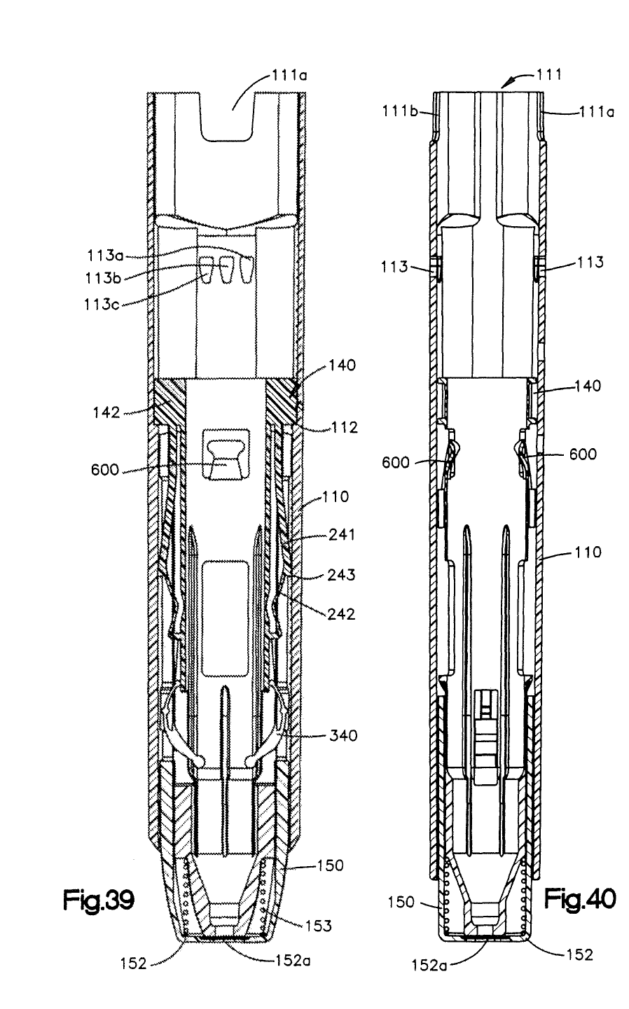

As shown in FIGS. 39, 41, 49, 50 and 52, the elongated body 141 of the cartridge container 140 includes a recess 244 located between the thinner strut 242. If the locking arms 240 are located on the outer body 110, the recess 244 could be formed in the outer body 110. Alternatively, an opening in the outer body 110 could also be provided. This recess 244 increases the distance that the thinner strut 242 travels inwardly toward the body 141, which increases the spring force provided to the thicker strut 241 to maintain the strut 241 in an outwardly biased position. The locking wings 240 are normally maintained in unstressed states. The locking wings 240 are compressed temporarily as the needle cover 150 passes over them. The locking wings 240 spring out such that the locking surface 243 engages the cover member 150 to prevent the needle cover 150 from being pushed backwards as shown in FIG. 10.

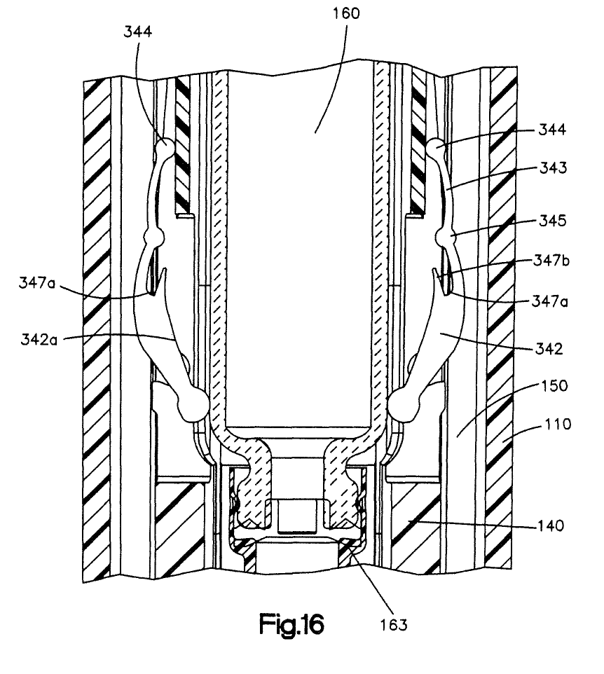

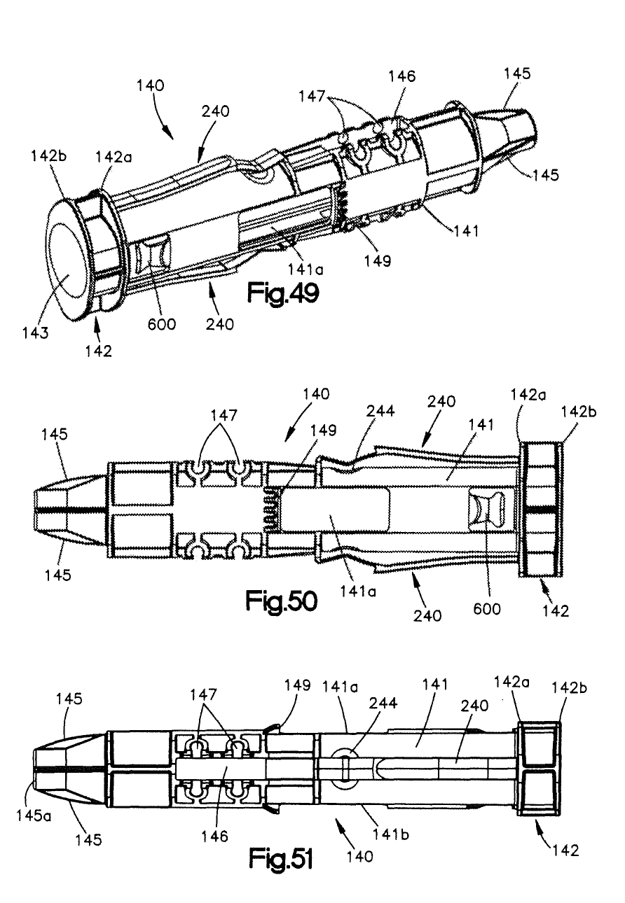

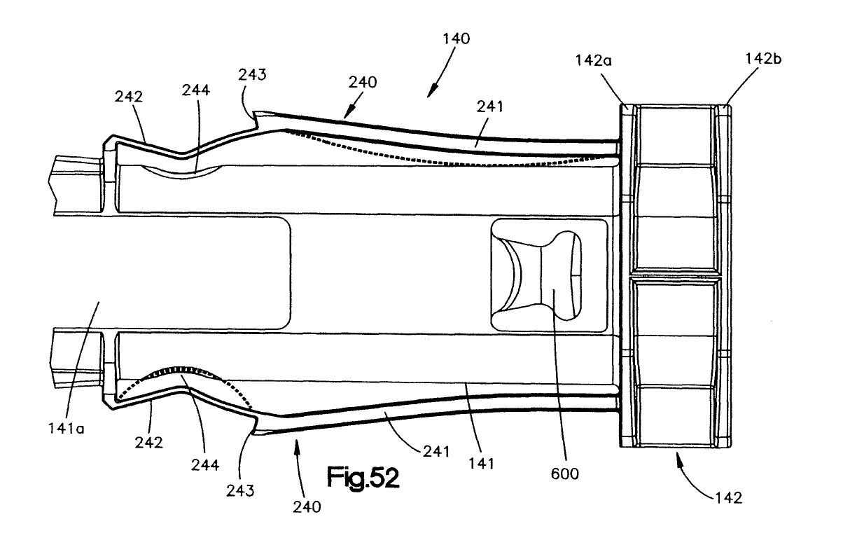

An elongated slot 146 is formed on each side of the elongated body 141. The slot 146 extends from the ends of the strut 242, as shown in FIGS. 49 and 51. Each slot 146 is sized to receive a locking tooth 340. As shown in FIGS. 1, 2, 4, 5, 7, 9, 16, 39 and 41, the locking teeth 340 are locked on opposing sides of the cartridge container 140. The locking teeth 340 are provided to hold back the needle cover 150 from deploying until after operation of the auto-injector 100. A pair of locking teeth 340 are provided. While not preferred, it is contemplated that a single locking tooth 340 can be employed.

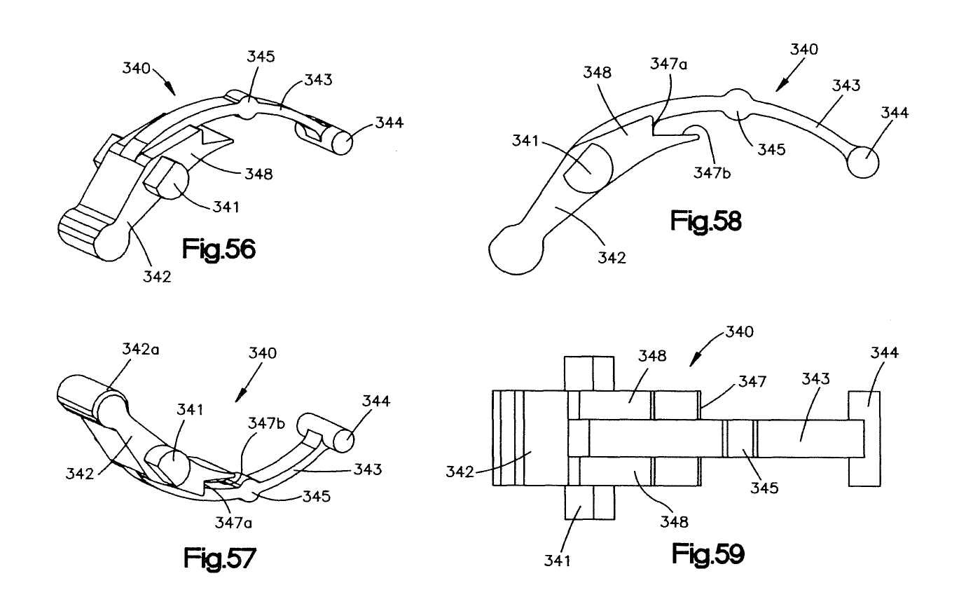

Each locking tooth 340 is capable of pivoting about the bearing axle 341 within the axle slot 147. Multiple axle slots may be provided such that the position of the tooth 340 may be adjusted. As shown in FIGS. 56-59, each locking tooth 340 has a tab 342 having a bearing surface 342a. The tab 342 is positioned within the slot 146 such that it extends into the interior of the elongated body 141 and is capable of contacting the cartridge 160. As the cartridge 160 is advanced within the body 141 during operation of the auto-injector 100, the contact between the cartridge 160 and the bearing surface 342a causes the locking tooth 340 to rotate about the axle 341. While the surface 342a contacts the cartridge 160, the locking teeth 340 have minimal or negligible impact on the movement of the cartridge 160 within the container 140 during the injection operation. The low or minimal force applied by the locking teeth to the cartridge is advantageous in that it does not build pressure within the cartridge that could prematurely burst the diaphragm before the needle is fully extended. Furthermore, the movement of the cartridge 160 within the container 140 is not impeded or negligibly impeded by the locking teeth 340. The tab 342 extends from one side of the axle 341. A spring tail 343 extends from an opposing side of the axle 341. The spring tail 343 is positioned within the slot 146 and is designed to slide along the cartridge container 140. The spring tail 343 serves to bias the locking tooth 340 into a locked position such that the needle cover 150 is retained or locked in a retracted position prior to operation of the auto-injector 100. It is contemplated that the spring tail 343 may be replaced with a spring assembly. A bearing surface 344 is provided on one end of the tail 343 to permit the spring tail 343 to slide smoothly along the cartridge container 140 within slot 146. The bearing surface 344 and central body 345 provide a flat area for an ejector pin.

Formed below the spring tail 343 is a v-shaped notch 347. The notch 347 has a locking surface 347a on one side which holds the needle cover 150 before activation of the auto-injector 100. Another surface 347b limits the travel of the tooth 340 within the cartridge container 140 to limit its rotation. The notch 347 is formed as part of a tab 348, which extends on either side of the spring tail 343. The locking teeth 340 increase the flexibility of the auto-injector 100. Numerous cartridges of various lengths and diameters can be used without modifying the auto-injector 100. The spring action of the tails 343 adjust the position of the locking teeth 340 such that the surface 342a contacts the cartridge 160.

The cartridge container 140 further includes a pair of openings 141a and 141b, which are formed on opposing sides of the body 141. The openings 141a and 141b permit viewing of the contents of the cartridge 160 such that the user can visually inspect the medicament prior to operation of the auto-injector 100. Prior to use the openings 141a and 141b are aligned with corresponding openings in the needle cover 150 such that the user can view the contents of cartridge 160 through the outer body 110. A ledge 149 having a plurality of reinforcing ribs 149a is formed adjacent one end of the opening 141. The ledge 149 contacts the edge 154a of the opening 154 in the needle cover 150 to prevent the needle cover 150 from moving any further forward relative to the cartridge container 140 so that the needle cover 150 cannot be pulled out of the outer body 110. When in this position, the locking surface 243 of the locking wings 240 engages the end of needle cover 150 to prevent the needle cover 150 from being inserted back into the outer body 110. When the ledge 149 is in contact with the edge of the opening in the needle cover 150, the openings in the cartridge container and the needle cover are no longer aligned such that the user cannot view the cartridge 160 through the outer body 110. This provides a visual guide indicator to the user that the auto-injector 100 has been used.

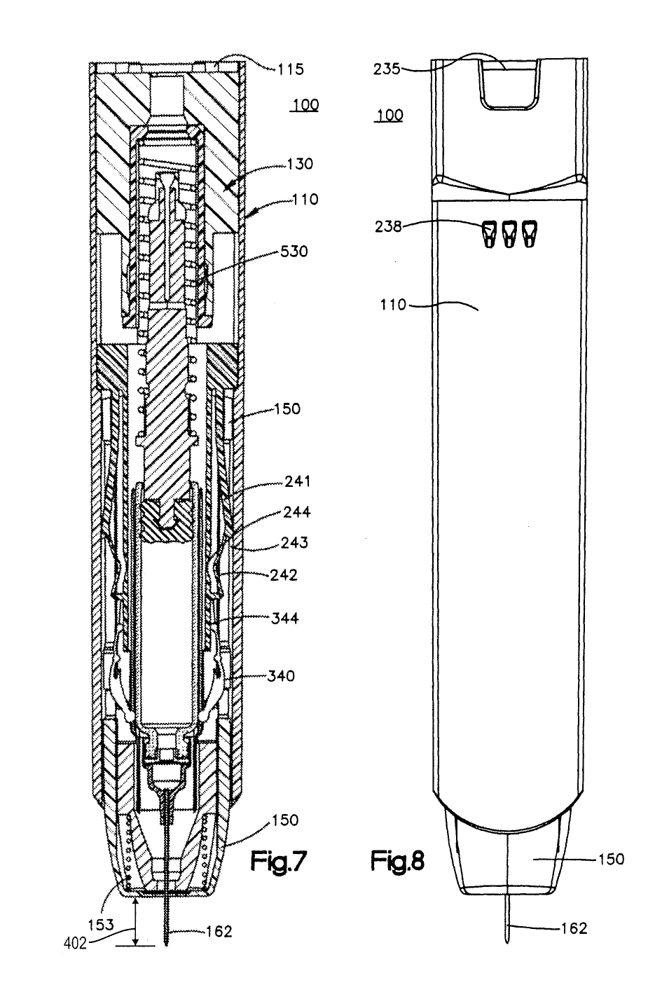

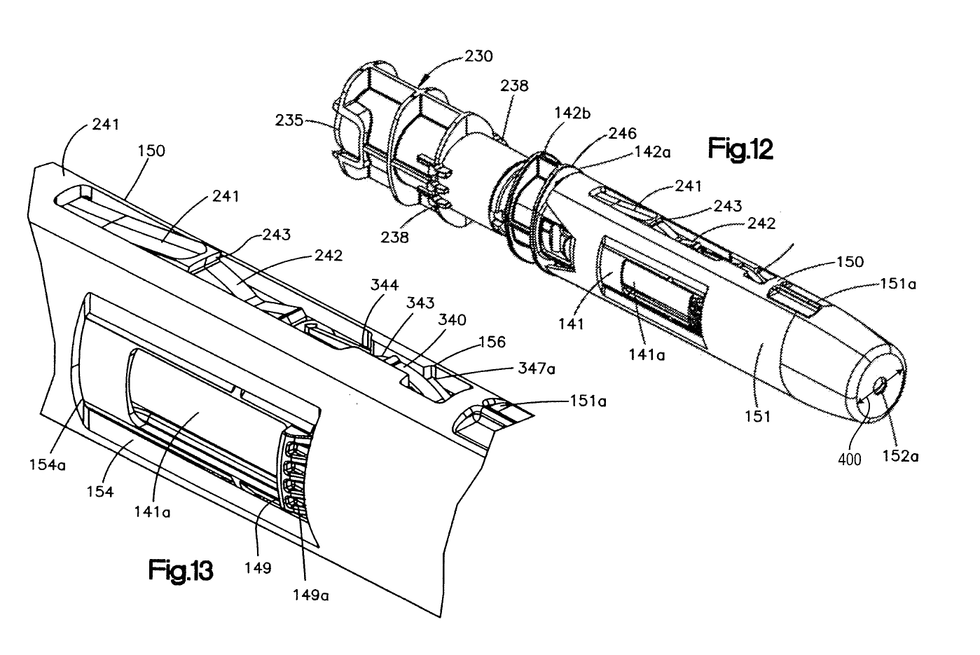

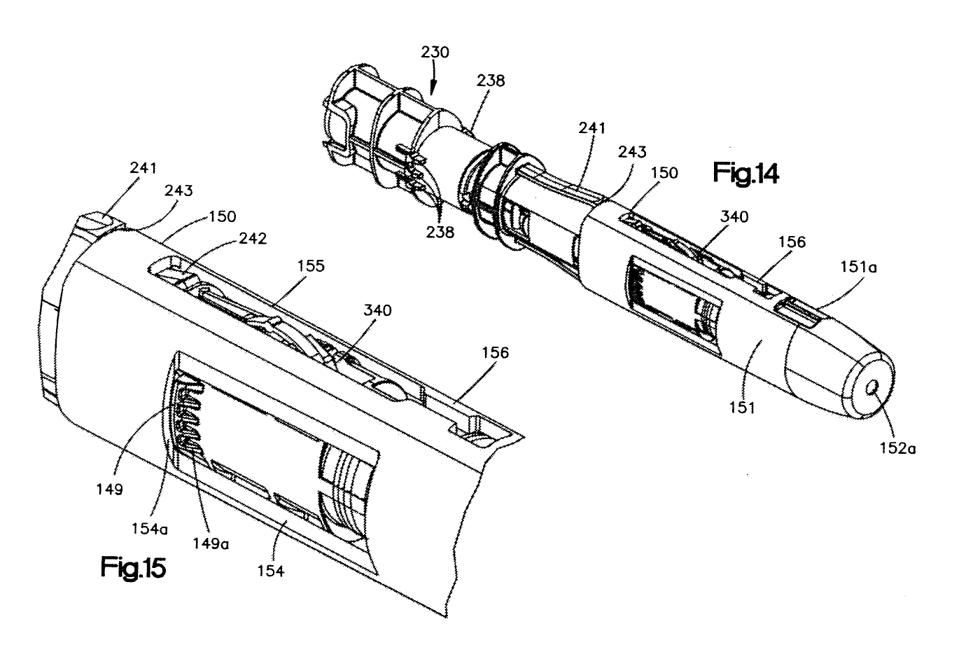

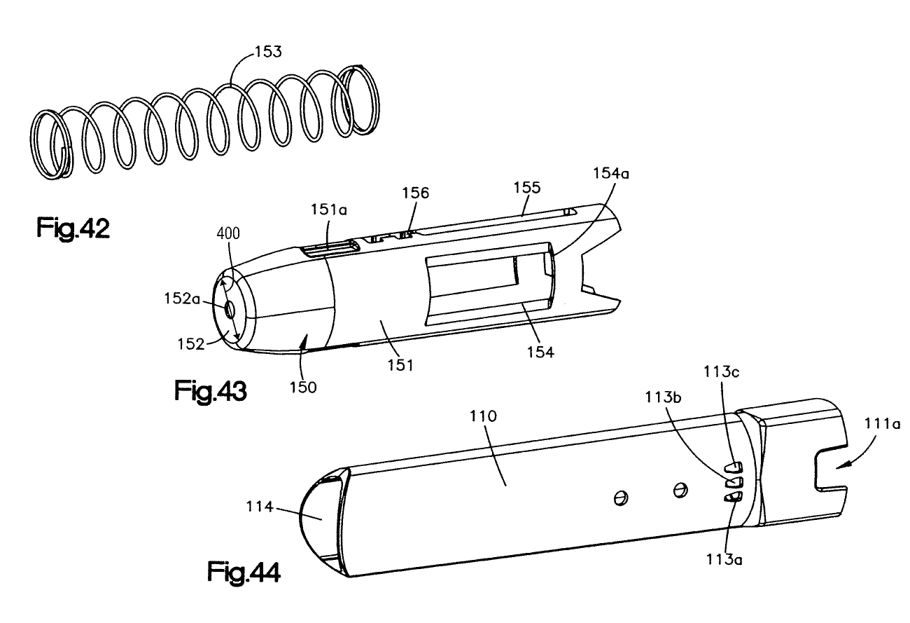

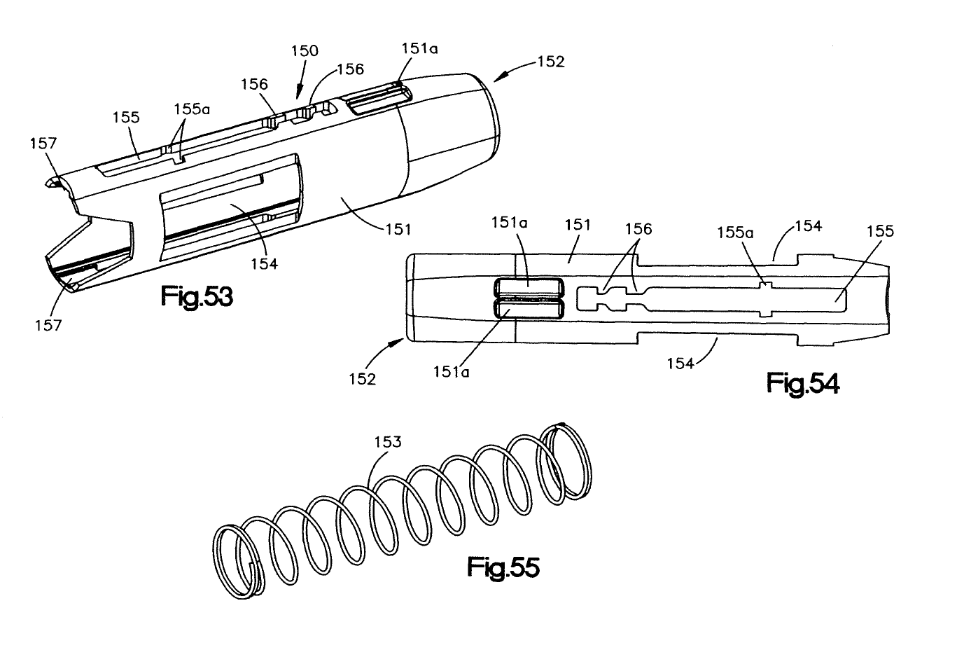

The needle cover 150 will now be described in greater detail in connection with FIGS. 12-15, 38, 42, 43 and 53-54. The needle cover 150 has a generally elongated hollow body 151 having a shape that is complementary to the shape of outer body 110. The elongated body 151 is slidably received within the outer body 100. One end of the hollow body 151 is tapered having an enclosed end surface 152. The end surface 152 has an opening 152a sized to permit the passage of the needle of the cartridge 160 there through during an injection operation, as shown in FIGS. 7 and 8. The end surface 152 is intended to be placed on the injection surface of the user during operation of the auto-injector 100 A needle cover spring 153 is compressed between the end surface 152 of the needle cover 150 and the cartridge container 140, as shown in FIGS. 1, 2, 4, 5, 7, and 9. The auto-injector 100 with needle cover 150 is designed to function like auto-injectors without needle covers in that a similar activation force is required to operate the auto-injector. As such, the spring 153 has a very low load. The biasing force for the cover 150 is less than the activating force of the auto-injector 100. The maximum load for the spring 153 is preferably 1.5 pounds. The load is lower than the activation force (1.5 versus 4-8) necessary to actuate the auto-injector 100 such that the needle cover 150 does not impact the operation of the auto-injector 100 when compared to injectors without covers such as disclosed in the '893 patent. The ribs 145 on the cartridge container 140 act to stabilize the spring 153 within the cover 150. The hollow body 151 may include indents 151a, shown in FIGS. 53 and 54. The indents 151a reduce the thickness of the plastic to conserve materials.

The hollow body 151 further includes a pair of openings 154 formed thereon. As discusses above, the openings 154 align with the openings 141a and 141b in the cartridge container 140 prior to activation to allow visibility of the medicament within the cartridge 160. Edge surface 154a of the opening 154 is designed to contact ledge 149 to prohibit further advancement of the needle cover 150.

Slots 155 are provided on opposing sides of the needle cover 150. The slots 155 are positioned to be aligned with the locking wings 240 and the locking teeth 340. The slots 155 guide and support the locking wings 240 prior to deployment of the needle cover 150. A cross slot 155a may be provided to aid in the assembly of the auto-injector 100 such that the locking teeth 340 can be inserted in place on the cartridge container 140 through slot 155 in the needle cover 150. Bearing surface 344 can be placed through the slot 155a. Locking projections 156 extend inwardly into the slot 155. The locking projections 156 are configured to engage the locking surface 347a on the locking teeth 340. Multiple projections 156 are provided to correspond to the multiple axle slots 147 in the cartridge container 140 for the bearing axle 341.

An interior groove 157 is provided within the interior of the hollow body 151. The interior groove 157 is axially aligned with the slots 155. A portion of the strut 241 is aligned in the groove 157 when the cover member 150 is in the position shown in FIGS. 12 and 13. The grooves are aligned with the locking wings 240 to provide support and prevent sideways collapsing of the locking wings 240.

The cartridge 160 includes a generally elongated glass tube having an opening 161 at one end sized to receive the plunger 438 and collet 430. The flange 436 on the collet 430 is designed to contact the end of the cartridge 160 to limit the inward travel of the plunger and collet into the cartridge 160 to control the dosage dispensed through the needle 162. The needle 162 is attached to a hub assembly 163 which is secured to another end of the cartridge 160. The hub assembly 163 may include a diaphragm 164 to prevent the passage of liquid medicament through the needle 162 prior to activation of the auto-injector. The needle 162 is encased in a protective sheath 165. The sheath 165 is secured to the hub assembly 163. The needle 162 pierces the sheath 165 during operation, when the needle 162 projects through the needle cover 150. The cartridge 160, as illustrated, provides a container for a dose of liquid medicament. It is not intended that the auto-injector 100 be limited solely to the use of a single liquid, rather, it is contemplated that one or more liquids may be stored in cartridge 160 that mix upon activation of the auto-injector 100. Furthermore, a solid medicament and a liquid can be separately stored in the cartridge 160 whereby the solid is dissolved in the liquid prior to dispensing.

The operation of the auto-injector 100 will now be described in greater detail. The auto-injector 100 is shown in an unactivated state in FIGS. 1, 2 and 3. The release pin 120 is secured in place such that the pin 125 is received within the hole 234 and the hole 435a in the collet 430 such that the side arms 433 cannot be inwardly deflected. In this position, the needle cover 150 is held in a locked retracted position by the locking teeth 340. The locking surfaces 347a are biased by the spring tails 343 into alignment with the locking projections 156 on the needle cover member 150. In this position, the auto-injector 100 cannot be operated and the needle 162 is not exposed.

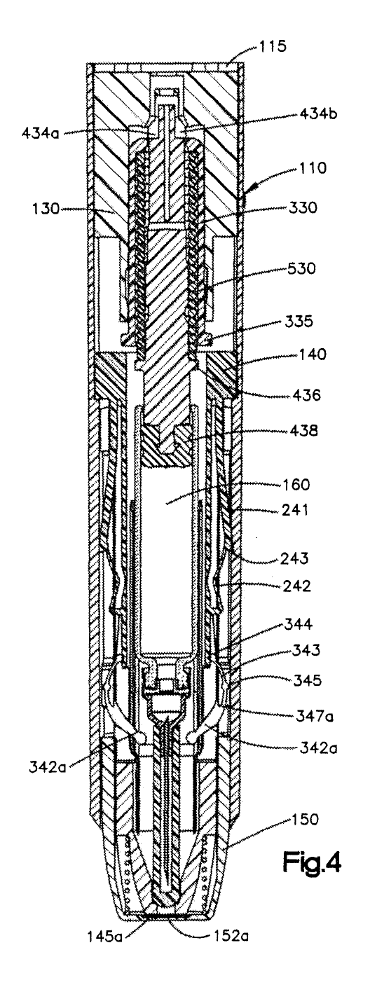

When operation of the auto-injector 100 is desired, the release pin 120 is grasped by the peripheral ledge 124 and pulled to remove the release pin 120 from the end of the auto-injector 100. This readies the auto-injector 100 for operation, as shown in FIG. 4. The arrowheads 434a and 434b and side arms 433a and 433b are now capable of being compressed together when the auto-injector 100 is activated. The locking wings 240 are not compressed or stressed at this time.

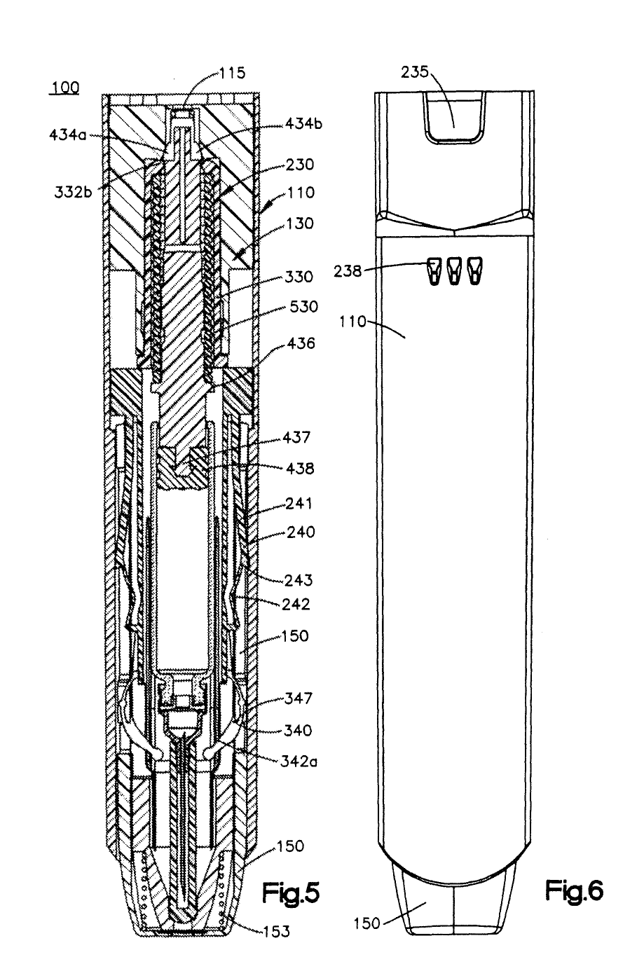

As shown in FIGS. 5 and 6, the user presses the end surface 152 of the needle cover 150 against the injection site. This causes the pre-compressed spring 153 to be slightly further compressed until the needle cover 150 moves and contacts the front end 145a of the cartridge container 140 (see FIG. 51), thus moving the ledge 142 of the cartridge container 140 rearwardly. The force of spring 153 is less that the force of spring 530. The needle cover 150, the cartridge container 140 and the cartridge 160 are then moved rearwardly into the outer body 110. The cartridge container 140 moves upward into the outer body 110 until the ledge 142 thereof contacts the ledge 335 of the power pack inner body 330. The power pack inner body 330, and the collet 430 and the spring assembly 530 are then pushed rearwardly into the auto-injector 100 into the power pack outer body 230. The collet 430 moves upwardly until it contacts the collet activation structure 239, shown in FIG. 28. The arrowheads 434a and 434b contact the sloped activation surface 239a. The arrowheads 434a and 434b are compressed together by the sloped surface 239 as the collet 430 moves rearwardly, such that the arrowheads 434a and 434b are released from the collet retention surface 332b. During this loading operation, the needle cover 150 is rearwardly pushed a small amount into outer body 110. When this occurs, the preload on the locking teeth 340 provided by the spring 153 is temporarily removed. As such, the v-shaped notch 347 temporarily disengages projection 156 formed on the needle cover 150. During this operation, the projection 156 no longer contacts either surface 347a or 347b, but remains in a space provided between the surfaces. As such, when pressure from the needle cover 150 is removed, the projection 156 will return into contact with the surfaces 347a or 347b. The locking teeth 340 will completely release the needle cover 150 only in response to movement of the cartridge 160 as it travels forwardly within the cartridge container 140. Accordingly, the needle cover 150 cannot deploy until the cartridge 160 moves.

The spring 530 and collet 430 simultaneously force the cartridge 160 and the cartridge container 140 forward toward the open front end of the outer body 110. Once the needle 162 has been extended through the needle cover 150, pressure of the medicament within the cartridge 160 causes the diaphragm 164 to burst permitting the flow of medicament into the user. The drug is forced through the needle 162 allowing the plunger 438 and collet 430 to move further into the cartridge 160. The cartridge container 140 retains the sheath 165 and also prevents the spring force of the spring 530 from being transferred through the cartridge 140 onto the needle cover 150 and the injection site. That is, the force from spring 530 that drives the cartridge 160 forward is opposed by the front end of the cartridge container 140, with the sheath 165 compressed there between, rather than force being received directly by the needle cover 150. In addition, the needle cover spring force is less than the activation force required to collapse the collet to release the collet during actuation. Preferably, the needle cover spring force is about 0.25 to 0.75 of the minimum activation force. The power pack residual spring force after activation is contained within the cartridge container 140, cartridge 160, the outer body 110 and the power pack outer body 230. This arrangement advantageously prevents a kickback effect from occurring. As such, the auto-injector is not pushed away from the injection site during activation to ensure that the proper dose of medicament is administered and the proper needle extended length or proper needle penetration is maintained. This effect would occur if the spring force from the spring 530 were transferred to the needle cover 150 and the injection site, whereby the auto-injector 100 could be pushed away from the injection site and alter the location of the needle 162 within the injection site. This has several negative impacts including startling the patient; changing the injection from an intramuscular to subcutaneous injection, which will affect pk levels. At the same time, the cartridge 160 is advanced within cartridge container 140 (i.e., when the needle 160 goes from a retracted position to extended position). The advancement of the cartridge 160 causes the locking tooth 340 to pivot about the axle 341. This is in response to cartridge 160 contacting bearing surface 342a and pushing the bearing surface 342a away from the main longitudinal axis of the needle 162. This rotation of the locking tooth 340 causes the locking surface 347a to disengage the locking projections 156. The surface 347b limits the rotation of the locking tooth 340. At this point, the cover member 150 is in an unlocked position such that it can move with respect to the cartridge container 140. The release of the collet 430 from the collet retention surface 332b forces the end of the power pack inner body 330 into contact with the power pack outer body 230.

Once the dose has been injected into the user, the user removes the auto-injector 100 from the injection surface. Since the needle cover 150 is not locked with respect to the cartridge container 140, the spring 153 forces the needle cover 150 out of the outer body 110 to cover the exposed needle 162, as shown in FIGS. 9 and 11. Since the slot 155 is aligned with groove 157 and a portion of the strut 241 is retained in the slot 157, the portion of the strut 241 moves into the groove 157 when the cover 150 moves outwardly. As the needle cover 150 slides outwardly, the locking wings 240 are temporarily compressed by the needle cover 150 as the thicker strut 241 slides through the groove 157. This compression occurs when the bottom surface of the groove 157 contacts the top surface of the strut 241. The wings 240 compress in the manner shown in the dashed lines in FIG. 52. Once the thicker strut 241 clears the groove 157 such that the wings 240 and needle cover 150 are in the position illustrated in FIGS. 10, 14 and 15, the locking surface 243 contacts the end of the needle cover 150 to prevent the needle cover from being reinserted into outer body 110. In the event that inward force is applied, the struts 241 and 242 compress such that the locking wing 240 is pressed against the body 141 of the cartridge container 140 such that the surface 243 remains engaged with the needle cover 150. This arrangement limits the inward travel of the needle cover 150. The ledge 149 engages the edge 154a of the opening 154 in the needle cover 150. The auto-injector 100 is now in an inoperable stored position.

### DIMENSIONAL EXAMPLES

The auto-injector construction described above may be embodied in specific articles of various dimensions, using components of various dimensions, and applied in use according to various operating parameters, for various purposes.

Those dimensional features and parameters of use, which may be the subject of selection for a particular purpose may include the end surface area of the needle cover, the force applied to the injection site by the end surface of the needle cover, the time interval over which the end surface of the needle cover is held in place against the injection site after injection, the volume of medicament to be delivered, the size of the internal passage through the needle, the injection depth as determined by the needle length protruding from the device, the spring force applied to the plunger to expel the medicament, and the time interval required for injection of the medicament upon actuation of the device. It is believed that these factors individually and/or collectively in various combinations, and possibly others, may contribute to the effectiveness of the device in delivering the medicament into the patient's body, and in dispersion of the medicament from the initial injection site into the surrounding bodily tissues, which may be referred to as the “uptake” of the medicament. One such other factor which may contribute to these results is the anti-kickback design of the auto-injector as described herein.

End Surface Area

The flat planar end surface area of the needle cover is illustrated in FIGS. 12 and 43. There the elongated hollow body 151 of the needle cover 150 is seen to include the enclosed end surface 152 having the end surface opening 152a formed therein to permit passage of the needle 162 there through. In the example shown, the enclosed end surface 152 is generally circular in shape, although it could be other shapes, for example elliptical or oval. In the example shown the elongated hollow body 151 tapers from an elliptical or oval cross-section shape in the portion thereof received in the housing 110 to the generally circular shape of the end surface 152. The surface area of the end surface 152 is generally annular in shape and is equal to the area contained within the outer diameter 400, minus the area of the end opening 152a. That surface area is preferably at least about 0.20 square inch, and more preferably at least about 0.24 square inch.

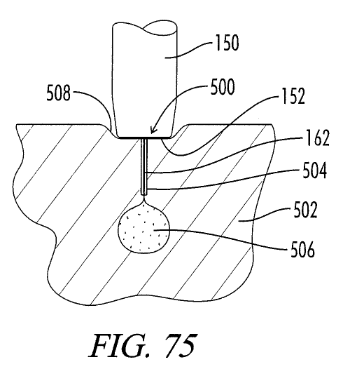

As is schematically illustrated in FIG. 75, at the injection site 500 the end surface 152 of the needle cover 150 compresses the skin, fat and muscle making up the user's body tissues 502. The compression of tissue contributes to a deeper penetration of the needle 162 into the user's body. The needle 162 creates a puncture passage 504 through the user's tissue, and a bolus 506 of medicament is injected into the user's body. One problem sometimes encountered in the use of auto-injectors is that there can be flow back of the injected medicament through the puncture passage 504 around the needle 162. With the auto-injector disclosed herein having the relatively large flat end surface 152 held in place against the user's body at the injection site, the bodily tissues are compressed as schematically indicated by the depressed area 508 on the surface of the user's skin. This compresses the skin, fat and muscle tissues around the needle 162 thus tending to seal the puncture passage 504 around the needle 162 and reducing flow back of the injected medicament out of the puncture passage.

Force Applied to Injection Site

The force applied to the injection site by the end surface 152 of the needle cover is ultimately determined by how hard the user chooses to press the device against the user's body, but a minimum level of that force is determined by the design of the device and the force required to actuate the device. As noted above in some embodiments the actuation force to release the energy stored in the power pack may be between 4 to 8 pounds. In other embodiments the actuation force to release the energy stored in the power pack may be between 2 to 8 pounds. The force actually applied by the user would therefore be at least about 2 pounds, and is more preferably at least about 4 pounds, and still more preferably at least about 5 pounds.

Hold Time after Injection

The time interval over which the end surface of the needle cover is held in place against the injection site after injection is typically based upon manufacturer's recommendations as printed upon the auto-injector label. For example a time interval of at least three seconds, or at least five seconds, or at least ten seconds may be recommended.

Volume of Medicament Injected

The volume of medicament to be delivered is dependent upon the internal dimensions of the device which are selected by the manufacturer to administer the desired volume of medicament. For example, when administering epinephrine with an auto-injector, an injected volume of about 0.15 mL or about 0.30 mL may be used. Higher volumes of injectant may also be administered. For example from 0.50 mL up 3.0 mL volumes may be rapidly injected, depending upon viscosity and other factors. As used herein, references to an injected or dispensed volume of medicament, are referring to the total volume of liquid injected into the patient, and those references are not related to the amount of active ingredient contained in that injected volume.

It will be appreciated that the amount of active ingredient in an injected volume of medicament may vary. The amount of active epinephrine ingredient in that injected volume may differ depending upon the dosage prescribed for the patient. Prescribed dosages of epinephrine active ingredient may for example be 0.075 mg, 0.15 mg, 0.3 mg or 0.5 mg. Those dosages of active ingredient may be formulated with other ingredients and water to comprise a volume of medicament of from 0.15 mL up to about 0.6 mL. The amount of active ingredient is not necessarily directly related to the volume of medicament to be injected, because the volume can be diluted as desired. For example, 0.30 mL of injected medicament might contain 0.15 mg or 0.30 mg of active epinephrine.

Needle Bore Size

The size of the internal passage through the needle is determined by the manufacturer by selection of the appropriate gauge of tubing for the needle 162. Small diameter stainless steel tubing typically used for hypodermic needles can be obtained in various standard sizes referred to as gauges. The gauge determines the nominal outside diameter of the tubing. Then for each gauge the tubing is typically available in various wall thickness referred to as Regular Wall (RW), Thin Wall (TW), Extra Thin Wall (ETW) and Ultra Thin Wall (UTW). The thinner the wall for a given gauge of tubing, the larger the internal diameter or bore of the tubing will be. And for each standard tubing size, such as for example a 22 gauge RW tubing, the applicable standards specify minimum, nominal and maximum values for each dimension such as the internal diameter. For the auto-injector construction described above, the needle 162 may be constructed from RW stainless steel tubing of gauges as large as 18 gauge and as small as 24 gauge. Wall thicknesses other than RW could also be selected. The following Table 1 shows standard minimum, nominal and maximum inner diameters for 18 to 24 gauge RW stainless steel tubing. All dimensions are given in inches.

TABLE 1

| GAUGE | TYPE | MIN I.D. | NOMINAL I.D. | MAX I.D. |
|---|---|---|---|---|
| 18 | RW | 0.0315 | 0.0330 | 0.0345 |
| 19 | RW | 0.0255 | 0.0270 | 0.0285 |
| 20 | RW | 0.0230 | 0.0238 | 0.0245 |
| 21 | RW | 0.0195 | 0.0203 | 0.0210 |
| 22 | RW | 0.0155 | 0.0163 | 0.0170 |
| 23 | RW | 0.0125 | 0.0133 | 0.0140 |
| 24 | RW | 0.0115 | 0.0123 | 0.0130 |

Thus, selecting the smallest of these inner diameters, the minimum inner diameter for the 24 gauge RW tubing is 0.0115 inch. If the 23 gauge RW tubing is selected, its inner diameter would be at least 0.0125 inch. If the 22 gauge RW tubing is selected, its inner diameter would be at least 0.0155 inch. For all of the selections shown in the above table, the inner diameter of the needle would be no greater than 0.0345 inch, which is the maximum inner diameter for an 18 gauge RW tubing.

Injection Depth

The injection depth as determined by the needle length protruding from the auto-injector is illustrated for example in FIG. 7 as the protruding length 402. That dimension is determined by the dimensions of the various internal components as is suitable for the particular medicament to be injected. For example, for subcutaneous injection of epinephrine the auto-injector may provide injections in the subcutaneous region wherein the injection is at a depth of from about 0.15 inches to about 0.30 inches, and more preferably of from about 0.2 inches to about 0.25 inches within the subject. On the other hand, for intramuscular injections of epinephrine the auto-injector may provide injections in the intramuscular region wherein the injection is at a depth of from about 0.4 inches to about 0.7 inches, and more preferably about 0.6 inches within the subject. For more obese patients, the auto-injector may provide intramuscular injections at a depth up to about 1.25 inches within the subject.

Spring Force

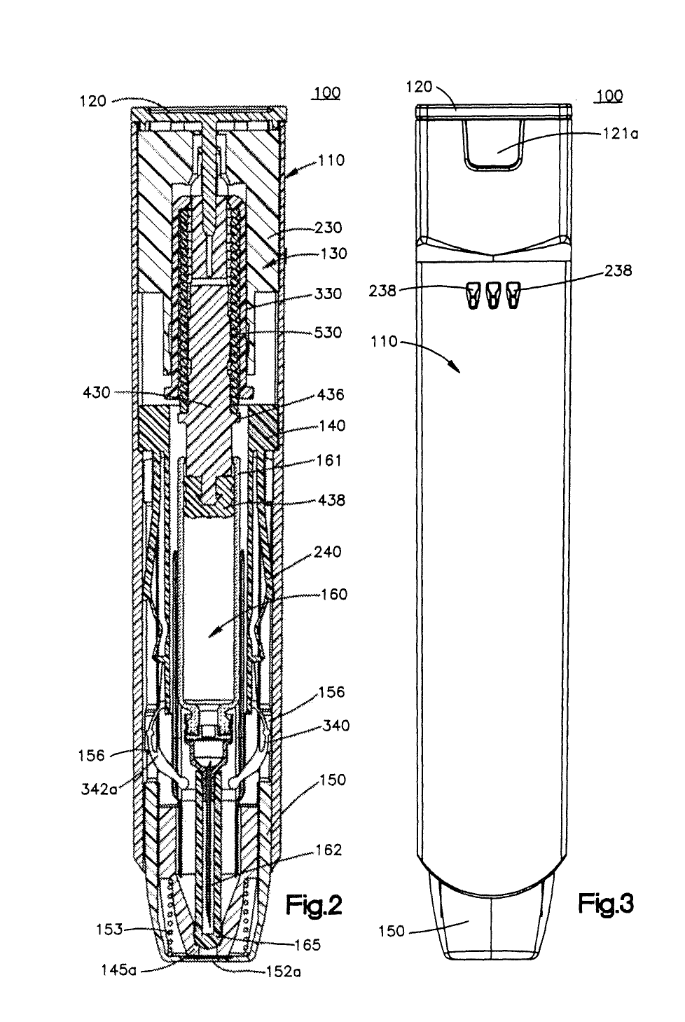

The spring force applied to the plunger to expel the medicament is determined by the manufacturer's selection of the power spring, and by the design of the various internal components which will affect how much of the available spring force is actually applied to the plunger. A given power spring 530, when compressed as seen in FIG. 2, will have a certain static force which it applies to the surrounding structure which holds the spring in the compressed state. When the spring 530 is released, however, some of its potential energy will be lost to friction and to moving the cartridge and needle and collapsing the needle sheath 165, so that the force the spring actually applies to the plunger 438 to expel the medicament, which can be referred to as a dynamic force applied to the plunger, will be less than the initial static force output of the spring 530. For example a spring 530 may have a nominal static force output of 30 pounds when compressed. Due to manufacturing tolerances the actual static force output of that spring may be in the range of from about 27 to about 33 pounds. Then the dynamic force that spring actually applies to the plunger 438 to expel the medicament may be in the range of from about 20 pounds to about 25 pounds. That dynamic force may be described as being at least about 20 pounds, and more preferably at least about 22 pounds.

Another factor of which the power spring force is a component is the dynamic action of the auto-injector, and particularly the dynamic action of the needle upon injection. It will be appreciated that the auto-injector is a complex spring, mass and dampener system which affects the motion of the various components of the auto-injector upon actuation. It has been observed in high speed motion photography that the auto-injector disclosed herein exhibits an axial oscillatory motion of the needle immediately after the needle is extended to its maximum injection depth. This motion occurs during the time that the injectant is being injected into the patient's body tissue. This motion is a dampened oscillation of the entire spring, mass and dampener system. It is believed that this oscillatory effect is significantly increased via the use of the high spring forces as disclosed herein in combination with the collapsible resilient rubber sheath 165. This oscillatory motion of the needle during injection may contribute to increased tissue disruption and subsequent enhanced injectant uptake by the patient's body tissue.

Time Interval for Injection

The time interval required for injection of the medicament upon actuation of the device will be dependent upon the selection of many of the dimensional factors discussed above, and upon others. For example, for the injection of a 0.30 mL volume of epinephrine, those factors may be selected to result in a time interval for injection of no more than about 0.5 second, and more preferably no more than about 0.4 second, and even more preferably no more than about 0.3 second.

### SPECIFIC EXAMPLES

Two specific examples of such devices which we have developed are marketed by Meridian Medical Technologies, Inc., the assignee of the present application, as the Truject EpiPen® and the Truject EpiPen® Jr.

For the Truject EpiPen® auto-injector the specific values for the various dimensions and operating factors discussed above are as follows:
the end surface 152 has an outside diameter 400 of about 0.58 inch and an end opening diameter of about 0.147 inch which results in a surface area of at least about 0.24 square inches;
the activation force to release the energy stored in the power pack is typically about 5.5 pounds;
the recommended hold time after injection is preferably at least ten seconds;
the volume of medicament delivered is about 0.30 mL, and it contains about 0.30 mg of active epinephrine ingredient;
the needle is a 22 gauge RW stainless steel needle having a nominal inner bore of 0.0163 inch;
the injection depth is about 0.6 inch;
the static spring force is nominally about 30 pounds, and the resulting dynamic spring force applied to the plunger and the medicament is about 22.7 pounds; and
the time interval for the initial injection of medicament is typically about 0.3 second.

For the Truject EpiPen® Jr. auto-injector the specific values for the various dimensions and operating factors discussed above are as follows:
the end surface 152 has an outside diameter 400 of about 0.58 inch and an end opening diameter of about 0.147 inch which results in a surface area of at least about 0.24 square inches;
the activation force to release the energy stored in the power pack is typically about 5.5 pounds;
the recommended hold time after injection is preferably at least ten seconds;
the volume of medicament delivered is about 0.30 mL, and it contains about 0.15 mg of active epinephrine ingredient;
the needle is a 22 gauge RW stainless steel needle having a nominal inner bore of 0.0163 inch;
the injection depth is about 0.5 inch;
the static spring force is nominally about 30 pounds, and the resulting dynamic spring force applied to the plunger and the medicament is about 23.6 pounds; and
the time interval for the initial injection of medicament is typically about 0.3 second.

The EpiPen® and EpiPen® Jr. devices have been the subject of some testing comparing their effectiveness to certain competitive devices as set forth in the following test summary.

### Test Summary

The study investigated, characterized and compared the injection patterns of the Anapen® 300 micrograms in 0.3 ml solution for injection (pre-filled syringe) Adrenaline (Epinephrine) Auto-Injector (Anapen® 300) vs. the EpiPen® (epinephrine) Auto-Injector 0.3 mg (EpiPen®), the Twinject® auto-injector (epinephrine injection, USP 1:1000) 0.15 mg (Twinject® 0.15 mL) vs. the EpiPen® Jr (epinephrine) Auto-Injector 0.15 mg (EpiPen® Jr), and the Twinject® auto-injector (epinephrine injection, USP 1:1000) 0.30 mg (Twinject® 0.30 mL) vs. the EpiPen® (epinephrine) Auto-Injector 0.3 mg (EpiPen®) using CT scans in a pig model. The purpose of the image analysis was to determine the respective initial volume of dispersion of injectate into the muscle tissue post-injection and the subsequent uptake of the injectate over a 15 minute time frame. Study 2010-001 was initiated on Mar. 1, 2010 and study 2010-02 was initiated on Jul. 21, 2010. This test summary describes animal care, study injections, CT imaging and analysis and provides study conclusions.

Three groups of four pigs were anesthetized prior to test and control article injections and CT imaging. All control and test article auto-injectors contained a non-sterile injectate of 0.75 mL water for injection mixed with 0.25 mL Omnipaque 300™ per 1 mL of injectate. The injectate solution was mixed as a single batch and all test and control auto-injectors were filled from this single batch. For all auto-injectors, spring force was defined as the force applied on the plunger at the moment the drug is being injected.

Four pigs in Group P1 were injected with test article #1 (Anapen® 300) in the right thigh and control article #1 (EpiPen®) in the left thigh. The Anapen® 300 auto-injector contained 0.3 mL of injectate and had a 27 ga.×0.3″ needle. The EpiPen® auto-injector contained 0.3 mL of injectate and had a 22 ga.×0.6″ needle. Two of the 4 pigs were injected through a pre-cut denim patch (3″W×4″L) which was stapled to the skin of the thigh over the injection site. Two of the pigs were injected directly through the skin of the thigh. The Anapen® 300 and EpiPen® activated at spring forces of approximately 2.1 vs. 23.0 lbs, respectively.

Four pigs in Group P2 were injected with test article #2 (Twinject® 0.15 mL) in the right thigh and control article #2 (EpiPen® Jr) in the left thigh. The Twinject® auto-injector contained 0.15 mL of injectate and had a 25 ga.×0.5″ needle. The EpiPen® Jr auto-injector contained 0.3 mL of injectate and had a 22 ga.×0.5″ needle. Two of the four (4) pigs were injected through a pre-cut denim patch (3″W×4″L), which was stapled to the skin of the thigh over the injection site. Two of the pigs were injected directly through the skin of the thigh. The Twinject® 0.15 mL and EpiPen® Jr activated at approximate spring forces of 6.5 vs. 23.0 lbs, respectively.

Four pigs in Group P3 were injected with test article #3 (Twinject® 0.30 mL) in the right thigh and control article #1 (EpiPen®) in the left thigh. The Twinject® 0.30 mL auto-injector contained 0.3 mL of injectate and had a 25 ga.×0.5″ needle. The EpiPen® auto-injector contained 0.3 mL of injectate and had a 22 ga.×0.6″ needle. Two of the 4 pigs were injected through a pre-cut denim patch (3″W×4″L) which was stapled to the skin of the thigh over the injection site. Two of the pigs were injected directly through the skin of the thigh. The Twinject® 0.30 mL and EpiPen® activated at spring forces of approximately 2-6 lbs vs. 23.0 lbs, respectively.

Serial CT images were performed at 11 time points/animal: 0, 1, 2, 3, 4, 5, 7, 9, 11, 13 and 15 minutes. Animals were euthanized after the 15 minute CT image and the skin/fat layer at each injection site was measured post-mortem. The auto-injectors were CT imaged, post-injection, for needle length. Study groups are shown below in Table 2.

TABLE 2
Study Groups

| | | | Delivery | | Volume | CT Time |
|---|---|---|---|---|---|---|
| Group | Formula | Denim | Device | Site | (mL) | Points (min) |
| P1 | 0.75 mL water | w/ n = 2 | EpiPen ® | Left | 0.3 mL | 0, 1, 2, 3, 4, 5, 7, |
| (n = 4) | for injection | | | thigh | | 9, 11, 13, 15 |
| | mixed with 0.25 mL | w/o n = 2 | Anapen ® | Right | 0.3 mL | |
| | Omnipaque | | 300 | thigh | | |
| P2 | 300 ™ per 1 mL | w/ n = 2 | EpiPen ® Jr | Left | 0.3 mL | 0, 1, 2, 3, 4, 5, 7, |
| (n = 4) | of injectate | | | thigh | | 9, 11, 13, 15 |
| | | w/o n = 2 | Twinject ® | Right | 0.15 mL | |
| | | | 0.15 mL | thigh | | |
| P3 | | w/ n = 2 | EpiPen ® | Left | 0.3 mL | 0, 1, 2, 3, 4, 5, 7, |
| (n = 4) | | | | thigh | | 9, 11, 13, 15 |
| | | w/o n = 2 | Twinject ® | Right | 0.3 mL | |
| | | | 0.30 mL | thigh | | |

CT scan calculations using the Analyze© 7.0 Software Suite was done on a per voxel basis. Therefore, in addition to a volume measure (in mm³) for each time interval, the mean and standard deviation of voxel intensities within the segmented object denoting each injection site was provided. The volume measure was in direct correlation to the dispersion and spread of the injectate within tissue. The mean and standard deviation of voxel intensities together provided a view of the spread of the injectate contrast agent (Omnipaque™) from the injection site and its subsequent uptake from tissue.

Study Group P1:

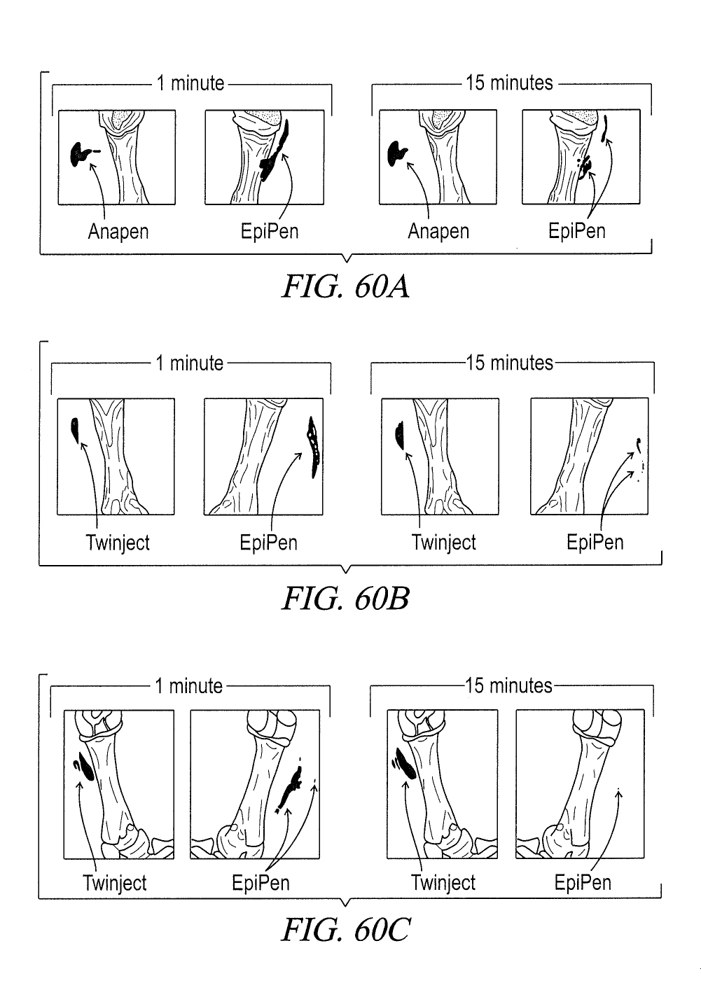

The larger average initial tissue dispersion volume (949.76 vs. 576.70 mm³), more rapid average peak dispersion volume (1 vs. 9 min.) and greater uptake of the injectate from the site of injection 15 minutes post-injection (80% vs. negligible) demonstrated that the EpiPen® auto-injector delivered injectate into muscle tissue with greater efficiency than the Anapen® 300 auto-injector in this study. As the injectate volumes between the two auto-injectors was identical (0.3 mL), it can be hypothesized that the greater delivery efficiency of EpiPen® auto-injector may be due to its larger needle size (22 ga. vs. 27 ga.), longer needle length (0.6″ vs. 0.3″) and/or greater spring force (approximately 23.0 vs. 2.1 lbs.), respectively. It was also noted that although the Anapen® 300 injectate volume was the same as the EpiPen®, it was delivered at different depths (0.3″ vs. 0.6″) and did not spread throughout the tissue over the 15 minute trial and remained essentially pooled. See example in FIG. 60A.

FIG. 60A shows EpiPen® vs. Anapen®300—Injectate Volume Visualization Using Analyze© at the 1 Min. Time Point (left) and 15 Min. Time Point (right) (Pig #105).

Study Group P2:

Greater peak injectate dispersion volume (934.77 vs. 412.07 mm³), more rapid average peak dispersion volume (1 vs. 7 min.) and greater uptake of the injectate 15 minutes post-injection (88% vs. negligible), demonstrated that the EpiPen® Jr delivered injectate into muscle tissue with greater efficiency than the Twinject® 0.15 mL. The auto-injector post-injection needle lengths were similar; however, other parameters of the EpiPen® and Twinject® 0.15 mL differed, such as: injectate volumes (0.3 mL vs. 0.15 mL), needle gauge (22 ga. vs. 25 ga.) and spring force (23.0 vs. 6.5 lbs), respectively. The greater delivery efficiency of EpiPen® Jr may therefore be a result of the larger needle size of the EpiPen® Jr and greater spring force. It was also noted that although the Twinject® 0.15 mL injectate volume was 50% of the EpiPen® Jr injectate volume, it was delivered at the same approximate depth (0.5″) but did not spread throughout the tissue over the 15 minute trial and remained essentially pooled. See example in FIG. 60B.

FIG. 60B shows EpiPen® Jr vs. Twinject® 0.15 mL—Injectate Volume Visualization Using Analyze© at the 1 Min. Time Point (left) and 15 Min. Time Point (right) (Pig #123).

Study Group P3:

Greater initial injectate dispersion volume (791.94 vs. 721.18 mm³), more rapid average peak dispersion volume (0 vs. 7-15 min.) and greater uptake of the injectate 15 minutes post-injection (97% vs. negligible), demonstrated that the EpiPen® delivered injectate into muscle tissue with greater efficiency than the Twinject® 0.30 mL. The auto-injector injection volumes and post-injection needle lengths were similar; however, other parameters of the EpiPen® and Twinject® 0.30 mL differed, such as: needle gauge (22 ga. vs. 25 ga.) and spring force (23.0 vs. 6.5 lbs), respectively. The greater delivery efficiency of EpiPen® may therefore be a result of the larger needle size and greater spring force. It was also noted that although the Twinject® 0.30 mL and EpiPen® injectate volumes were the same and were delivered at similar depths (0.5″ vs. 0.6″), Twinject® 0.30 mL injectate uptake remained negligible at the 15 minute time point. See example in FIG. 60C.

FIG. 60C shows EpiPen® vs. Twinject® 0.30 mL—Injectate Volume Visualization Using Analyze© at the 1 Min. Time Point (left) and 15 Min. Time Point (right) (Pig # XX).

Test Article Comparison:

The Twinject® 0.30 mL auto-injector demonstrated a larger initial injectate dispersion volume (721.18 mm³) vs. the Twinject® 0.15 mL (412.04 mm³) and the Anapen® 300 (576.70 mm³) and reached peak injectate dispersion volume more slowly (15 min. vs. 7 and 9 min. respectively). This data suggests that the Twinject® 0.30 mL dispersed injectate more widely but reached its peak volume more slowly than either of the other test articles. The dispersion difference observed between the two types of Twinject® auto-injectors could be explained by the larger volume of the Twinject® 0.30 mL vs. the Twinject® 0.15 mL. The dispersion difference between the Twinject® 0.30 mL vs. the Anapen® 300 auto-injectors could be explained by the larger needle gauge and the greater spring force of the Twinject® 0.30 mL vs. the Anapen® 300 when dispensing equal volumes of injectate. None of the three test articles displayed appreciable uptake of injectate, either in general or relative to one another, suggesting that these auto-injectors did not effectively deliver injectate in a manner that led to uptake within the muscle tissue.

Control Article Comparison:

The EpiPen® auto-injector (Group P1) and (Group P3) reached peak injectate dispersion volumes of 955.84 mm³ and 791.94 mm³ at one (1) min. and zero (0) min., respectively. The EpiPen® Jr (Group P2) reached peak injectate dispersion volume (934.77 mm³) at zero (0) min. The injection volume and spring force of the EpiPen® and EpiPen® Jr were the same (0.3 mL and 23.0 lbs, respectively). This data suggests uniformity between the control articles in injectate tissue dispersion. Both control articles displayed appreciable uptake of injectate. The difference in injectate uptake volume seen at the 15 minute time point (EpiPen®—80% (Group P1) and 97% (Group P2) vs. EpiPen® Jr—88%) was not significant, as it was within the variance noted within each trial.

Denim Patch vs. Skin Injections:

There were no appreciable differences in either injectate dispersion or pattern of injectate uptake for test (Anapen® 300, Twinject® 0.15 mL or Twinject® 0.30 mL) or control article (EpiPen® or EpiPen® Jr) injections with respect to whether the article was applied through denim or directly through the skin. It is noteworthy that data was analyzed with only two (2) animals per group. However, these data show that all auto-injector needles were able to successfully penetrate the denim and injections through denim did not appear to affect any dispersion or uptake of study injectate.

Post-Injection Needle Lengths:

All 24 test and control article post-injection needle lengths approximated the needle lengths claimed on their respective labels. The variance of the three needle measurements is within the lower bounds of measurement resolution using this CT analysis method.

Post-Mortem Injection Site Skin/Fat Layer Measurement:

The average measure of the skin/fat layer of injections sites ranged between 1.65-3.57 mm, averaging 2.23 mm. This data showed that auto-injections were given into muscle through a relatively uniform thickness of the skin/fat layer in all animals.

### Conclusion Summary

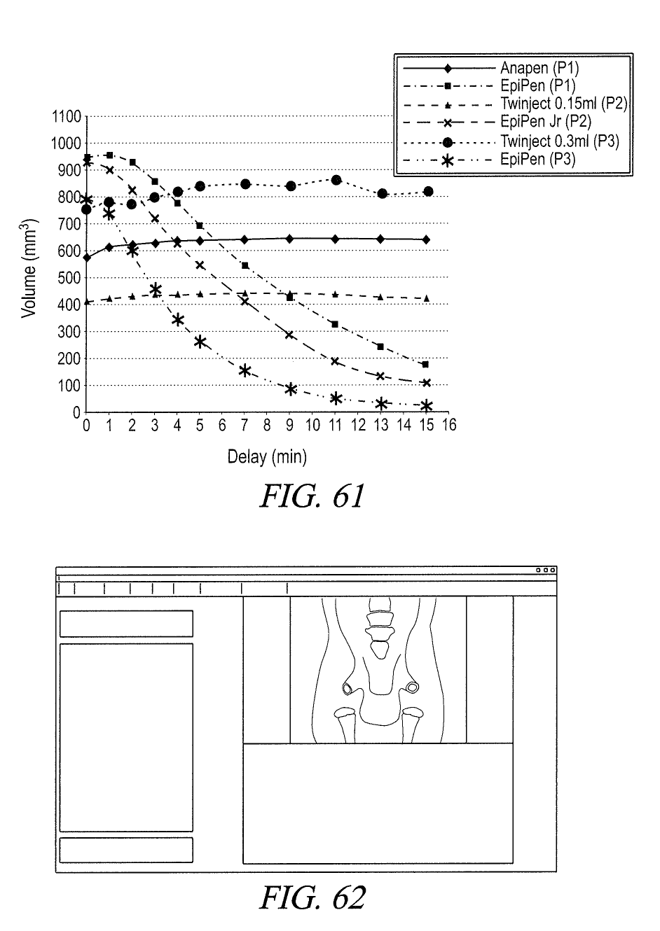

The control article auto-injectors (EpiPen® and EpiPen® Jr) delivered injectate into the muscle tissue with greater efficiency than the test article auto-injectors (Anapen® 300, Twinject® 0.15 mL and Twinject® 0.30 mL). This efficiency was demonstrated by larger tissue dispersion volumes, more rapid peak dispersion volume and greater uptake of the injectate at the 15 minute post injection time point. Additionally, there was similarity in the pattern of injectate uptake between the EpiPen® and EpiPen® Jr auto-injectors and in the end injectate volume of uptake (80 and 97% vs. 88%, respectively). In contrast, while the Twinject® 0.30 mL auto-injector demonstrated a larger dispersion volume than either the Twinject® 0.15 mL or the Anapen® 300, none of the test articles displayed appreciable uptake of injectate, either in general or relative to one another, and remained essentially pooled in the tissue at the 15 minute time point as shown in FIG. 61. FIG. 61 is a comparison of Test and Control Article Auto-injections—Group P1 (Anapen® 300 and EpiPen®), Group P2 (Twinject® 0.15 mL and EpiPen® Jr) and Group P3 (Twinject® 0.30 mL and EpiPen®) (Pig #105-108, 120-123, #229-232).

### 2.0 Introduction

The aim of this study was to investigate, characterize and compare the injection patterns of the Anapen® 300 micrograms in 0.3 ml solution for injection (pre-filled syringe) Adrenaline (Epinephrine) Auto-Injector (Anapen® 300) vs. the EpiPen® (epinephrine) Auto-Injector 0.3 mg (EpiPen®), the Twinject® auto-injector (epinephrine injection, USP 1:1000) 0.15 mg (Twinject® 0.15 mL) vs. the EpiPen® Jr (epinephrine) Auto-Injector 0.15 mg (EpiPen® Jr), and the Twinject® auto-injector (epinephrine injection, USP 1:1000) 0.30 mg (Twinject® 0.30 mL) vs. the EpiPen® (epinephrine) Auto-Injector 0.3 mg (EpiPen®) using CT scans in a pig model. The Georgetown University Medical Center protocol study numbers were 2010-001 and 2010-02. The studies were initiated in the Division of Comparative Medicine on Mar. 1, 2010 and Jul. 24, 2010 under an approved animal care and use protocol (#10-005). Live animal activities were conducted by Beverly Jan Gnadt, DVM, DACLAM; Robin Tucker, DVM, DABT; April Yancy, DVM, MPH; Jenna Hargens, BS, RLAT; Elizabeth Probst, BS, RLAT, LVT; Rebecca Lossing, BS, MS; Bennie Johnson, BS, RALAT and Amanda Thress, AA, RVT. CT scans and evaluations were conducted by Kevin Cleary, PhD; Filip Banovac, MD; Emmanuel Wilson, MS; David Lindisch, RT; and George Armah, RT.

This study was not subject to the requirements set forth in the FDA Good Laboratory Practices, 21 CFR Part 58; however, the studies were conducted in the spirit of the GLP guidelines to the extent possible.

### 3.0 Materials and Methods

### 3.1 Animals

Thirteen (13) female Yorkshire pigs were purchased from Thomas D. Morris, Inc. (Reisterstown, MD) and shipped to Georgetown University, Division of Comparative Medicine. One (1) animal arrived on Feb. 25, 2010, four (4) animals on Mar. 3, 2010, four (4) animals on Mar. 17, 2010 and four (4) animals on Jul. 21, 2010. The animals were identified at the vendor using permanent ear tags (#22, 105,106,107,108,120,121,122,123, 229, 230, 231 and 232). One pre-study pig (ear tag #22) was euthanized the day after arrival and was used to determine the specific location for study injection sites and the optimal size/placement/attachment method of denim patches on the skin at the injection site. The remaining twelve (12) pigs were used on the main study.

Animals were visually assessed on arrival, weighed upon receipt and assigned study numbers (Table 3). All animals were housed in pens with raised floors. The pre-study pig (ear tag #22) was housed singly and used the day after arrival. The twelve main study animals were gang housed for a minimum of three (3) days during the acclimation period. Animals were also individually housed per veterinary decision.

TABLE 3
Animal Study Numbers and Weights on Receipt

| | Pig Ear | | | |
|---|---|---|---|---|
| | Tag | Date of | | Body Weight |
| Pig Study Number | Number | Receipt | | (Kg) |
| NA | 22 | Feb. 25, | 2010 | 32.8 |
| 2010-001-105-P1-D | 105 | Mar. 3, | 2010 | 32.4 |
| 2010-001-106-P1-ND | 106 | Mar. 3, | 2010 | 30.6 |
| 2010-001-107-P1-D | 107 | Mar. 3, | 2010 | 30.6 |
| 2010-001-108-P1-ND | 108 | Mar. 3, | 2010 | 30.6 |
| 2010-001-120-P2-D | 120 | Mar. 17, | 2010 | 32.2 |
| 2010-001-121-P2-ND | 121 | Mar. 17, | 2010 | 32.8 |
| 2010-001-122-P2-ND | 122 | Mar. 17, | 2010 | 31.3 |
| 2010-001-123-P2-D | 123 | Mar. 17, | 2010 | 34.0 |
| 2010-02-229-P3-ND | 229 | Jul. 21, | 2010 | 33.7 |
| 2010-02-230-P3-D | 230 | Jul. 21, | 2010 | 30.4 |
| 2010-02-231-P3-D | 231 | Jul. 21, | 2010 | 31.5 |
| 2010-02-232-P3-ND | 232 | Jul. 21, | 2010 | 28.8 |

Animals were fed Purina™ Lab Diet 5084 (non-certified) twice daily. Tap water, provided by an automatic water system, was available ad libitum from the day of arrival to the end of study. The study director and sponsor considered possible interfering substances potentially present in animal feed and water. There was no reasonable expectation that any contaminant was present in the feed or that any component of the feed affected the Omnipaque 300™ injectate solution distribution. Facility water was pre-filtered before being supplied through the automatic watering system. Routine water analysis for chemical and microbiological contamination is performed annually. Based on previous testing results, no contaminants were reasonably expected to be present in water at levels sufficient to interfere with the study.

Animal room environmental temperatures were targeted between 68-81° F. and between 30-70% relative humidity. Temperature and humidity were monitored continuously by a chart recorder (Dickson TH6 Chart recorder), except when interrupted for study related events. A 12-hour light/12-hour dark cycle was maintained, except when interrupted for study related events. Ten or greater air changes per hour were maintained. Animals were provided food and enrichment during acclimation.

Animals were visually observed daily for mortality, morbidity, general health and food consumption. All animals were examined by a veterinarian and were found to be in suitable health for use on study. Animals were visually observed again prior to test and control article administration.

### 3.1.1 Randomization, Group Designation and Dosage Levels

One pig (ear tag #22) was used for pre-study procedural assessment and was not entered into either main study group.

For the twelve (12) remaining animals, cards were labeled with group designations P1, P2 and P3 (4 cards per group). Four (4) P1 animals were to receive test article #1—Anapen® 300, four (4) P2 animals were to receive test article #2-Twinject® 0.15 mL and four (4) P3 animals were to receive test article #3—Twinject® 0.30 mL. Each set of four (4) animals were randomized into denim (two (2) animals) vs. no denim (two (2) animals) groups by random card draw.

The P1 study group utilized Anapen® 300 as the test article (27 ga.×0.3″ needle) and EpiPen® as the control article (22 ga.×0.6″ needle). These four animals received two simultaneous 0.3 mL injections intramuscularly. The test article was injected into the right thigh muscle and the control article was injected into the left thigh muscle.

The P2 study group utilized Twinject® 0.15 mL auto-injectors as the test article (25 ga.×0.5″ needle) and EpiPen® Jr auto-injectors as the control article (22 ga.×0.5″ needle). The Twinject® 0.15 mL auto-injectors delivered 0.15 mL and the EpiPen® Jr auto-injectors delivered 0.3 mL. These four animals received simultaneous injections intramuscularly. The test article was injected into the right thigh muscle and the control article was injected into the left thigh muscle.

The P3 study group utilized Twinject® 0.30 mL auto-injectors as the test article (25 ga.×0.5″ needle) and EpiPen® auto-injectors as the control article (22 ga.×0.6″ needle). The Twinject® 0.30 mL auto-injectors delivered 0.30 mL and the EpiPen® auto-injectors delivered 0.3 mL. These four animals received simultaneous injections intramuscularly. The test article was injected into the right thigh muscle and the control article was injected into the left thigh muscle.

See study groups P1, P2 and P3 in Table 4 below.

3.1.2 Study Groups

TABLE 4
Study Groups (P1, P2 and P3)

| | | | Delivery | | Volume | CT Time Points |
|---|---|---|---|---|---|---|
| Group | Formula | Denim | Device | Site | (mL) | (min) |
| P1 | 0.75 mL water for | w/ n = 2 | EpiPen ® | Left | 0.3 mL | 0, 1, 2, 3, 4, 5, 7, |
| (n = 4) | injection mixed | | | thigh | | 9, 11, 13, 15 |
| | with 0.25 mL | w/o n = 2 | Anapen ® | Right | 0.3 mL | |
| | Omnipaque | | 300 | thigh | | |
| P2 | 300 ™ per 1 mL of | w/ n = 2 | EpiPen ® Jr | Left | 0.3 mL | 0, 1, 2, 3, 4, 5, 7, |
| (n = 4) | injectate | | | thigh | | 9, 11, 13, 15 |
| | | w/o n = 2 | Twinject ® | Right | 0.15 mL | |
| | | | 0.15 mL | thigh | | |
| P3 | | w/ n = 2 | EpiPen ® | Left | 0.3 mL | 0, 1, 2, 3, 4, 5, 7, |
| (n = 4) | | | | thigh | | 9, 11, 13, 15 |
| | | w/o n = 2 | Twinject ® | Right | 0.3 mL | |
| | | | 0.30 mL | thigh | | |

3.2 Test and Control Articles

3.2.1 Test Article Description

Test Article (Group P1)—

The Anapen® 300 is a round, pre-filled needle syringe combination designed to inject a single, pre-measured dose of medication into the thigh muscle. The Anapen® 300 needle is 27 gauge and extends approximately 0.3″ in length during injection. The Anapen® 300 activates at a spring force of approximately 2.1 lbs. with spring force defined as the force applied on the plunger at the moment the drug is being injected. To activate the Anapen® 300, the black ‘boot’ needle sheath remover at the base of the device is pulled off by gripping it firmly and pulling gently outward. Removing the black boot will extract the grey needle sheath, exposing the needle. The black safety cap is removed from the top of the device, exposing the red activation button. The device is gently but firmly placed against the thigh, ensuring that the red activation button is away from the thigh. The device is held steady and the red button is pressed only when ready to inject, as the button is quite sensitive. A ‘click’ is heard at the moment of injection. The device is held in place for 10 seconds to deliver all the medication. After automatic administration of the dose, the needle is exposed upon removal from the thigh muscle.

Test Article (Group P2)—

The Twinject® 0.15 mL is a round, pre-filled automatic syringe designed to inject a single, pre-measured dose of medication into the thigh muscle. The Twinject® 0.15 mL needle is 25 gauge and extends approximately 0.5″ in length during injection. The Twinject® 0.15 mL activates at a spring force of approximately 6.5 lbs. with spring force defined as the force applied on the plunger at the moment the drug is being injected. The Twinject® 0.15 mL has a delivered volume of 0.15 mL. It also stores a second pre-filled dose of 0.15 mL in the form of a manual syringe that a patient or caregiver can administer. To activate the first dose of the product, both green caps are pulled off in numerical order and the exposed red tip is pressed hard against the thigh until the auto-injector fires. The device is held in place for 10 seconds to deliver all the medication (0.15 mL). After automatic administration of the dose, the needle is exposed upon removal from the thigh muscle.

Administration of the second dose did not occur in this study, and this study only evaluated the initial spring-driven dose delivered by Twinject® 0.15 mL.

Test Article (Group P3)—

The Twinject® 0.30 mL is a round, pre-filled automatic syringe designed to inject a single, pre-measured dose of medication into the thigh muscle. The Twinject® 0.30 mL needle is 25 gauge and extends approximately 0.5″ in length during injection. The Twinject® 0.30 mL activates at a spring force of approximately 6.5 lbs. with spring force defined as the force applied on the plunger at the moment the drug is being injected. The Twinject® 0.30 mL has a delivered volume of 0.30 mL. It also stores a second pre-filled dose of 0.30 mL in the form of a manual syringe that a patient or caregiver can administer. To activate the first dose of the product, both green caps are pulled off in numerical order and the exposed red tip is pressed hard against the thigh until the auto-injector fires. The device is held in place for 10 seconds to deliver all the medication (0.30 mL). After automatic administration of the dose, the needle is exposed upon removal from the thigh muscle.

Administration of the second dose did not occur in this study, and this study only evaluated the initial spring-driven dose delivered by Twinject® 0.30 mL.

3.2.2. Control Article Description

Control Article (Groups P1 and P3)—

The EpiPen® is an oval, spring-driven, pressure activated, pre-filled automatic syringe. The EpiPen® needle is 22 gauge and extends approximately 0.6″ in length during injection. The EpiPen® activates at a spring force of approximately 23.0 lbs. with spring force defined as the force applied on the plunger at the moment the drug is being injected. The EpiPen® is equipped with a blue safety release to prevent accidental activation. The needle end of the EpiPen® is orange and is located on the end opposite the blue safety release. Once the safety release has been removed, the injection dose is administered by firmly pressing the flat orange face of the auto-injector against the injection site. Upon activation, a hypodermic needle extends rapidly from the center of the flat face of the orange end. The injectate is administered once the needle has reached full extension. Once activated, the EpiPen® should be held firmly in place for 10 seconds to ensure the injectate dose is completely injected.

The TruJect-style EpiPen® Auto-Injector is equipped with an automatically deployed sharps cover. Upon activation of the auto-injector and removal from the injection site, the orange nose extends from the auto-injector, locks into place and provides protection from the needle.

Control Article (Group P2)—

The EpiPen® Jr is an oval, spring-driven, pressure activated, pre-filled automatic syringe. The EpiPen® Jr needle is 22 gauge and extends approximately 0.5″ in length during injection. The EpiPen® Jr activates at a spring force of approximately 23.0 lbs. with spring force defined as the force applied on the plunger at the moment the drug is being injected. Each EpiPen® Jr is equipped with a blue safety release to prevent accidental activation. The needle end of the EpiPen® Jr is orange and is located on the end opposite the blue safety release. Once the safety release has been removed, the injection dose is administered by firmly pressing the flat orange face of the auto-injector against the injection site. Upon activation, a hypodermic needle extends rapidly from the center of the flat face of the orange end. The injectate is administered once the needle has reached full extension. Once activated, the EpiPen® Jr should be held firmly in place for 10 seconds to ensure the injectate dose is completely injected.

The TruJect-style EpiPen® Auto-Injector is equipped with an automatically deployed sharps cover. Upon activation of the auto-injector and removal from the injection site, the orange nose extends from the auto-injector, locks into place and provides protection from the needle.

3.2.3 Test Article Receipt and Internal Number Assignment

A total of 54 test article auto-injectors containing Omnipaque 300™ (0.75 mL water for injection mixed with 0.25 mL Omnipaque 300™ per 1 mL injectate) solution were received by the Division of Comparative Medicine (Georgetown University) from Meridian Medical Technologies, Inc. (Columbia, Md.) (Table 5). Testing facility personnel assigned internal numbers to test articles on the day of arrival. The Twinject® 0.3 mL auto-injectors were only used in the 2010-02 study.

TABLE 5
Test Article Receipt and Internal Number Assignment

| Type | Receipt Date | | Lot # | # Received | Test Article ID #'s |
|---|---|---|---|---|---|
| Anapen ® 300 | Feb. 5, | 2010 | FMY | 3 | 2010-001-AN-1 thru |
| | | | | | 2010-001-AN-3 |
| Anapen ® 300 | Feb. 16, | 2010 | FMY | 3 | 2010-001-AN-4 thru |
| | | | | | 2010-001-AN-6 |
| Anapen ® 300 | Feb. 25, | 2010 | FMY | 12 | 2010-001-AN-7 thru |
| | | | | | 2010-001-AN-18 |
| Twinject ® 0.3 mL | Feb. 5, | 2010 | U081201A | 3 | 2010-001-TW-1 thru |
| | | | | | 2010-001-TW-3 |
| Twinject ® 0.3 mL | Feb. 16, | 2010 | U081201A | 3 | 2010-001-TW-4 thru |
| | | | | | 2010-001-TW-6 |
| Twinject ® 0.3 mL | Feb. 25, | 2010 | U081201A | 12 | 2010-001-TW-7 thru |
| | | | | | 2010-001-TW-18 |
| Twinject ® 0.15 mL | Mar. 15, | 2010 | U08113C | 12 | 2010-001-TWJ-1 thru |
| | | | | | 2010-001-TWJ-12 |
| Twinject ® 0.3 mL | Jul. 22, | 2010 | XXX | 6 | 2010-02-TW-1 thru |

2010-02-TW-6

3.2.4. Control Article Receipt and Internal Number Assignment

A total of 42 control auto-injectors containing Omnipaque 300™ (0.75 mL water for injection mixed with 0.25 mL Omnipaque 300™ per 1 mL injectate) solution were received by the Division of Comparative Medicine (Georgetown University) from Meridian Medical Technologies, Inc. (Columbia, Md.). Testing facility personnel assigned internal numbers to test articles on the day of arrival. See Table 6 below.

TABLE 6
Control Article Receipt and Internal Number Assignment

| | | | | # Re- | Test |
|---|---|---|---|---|---|
| Type | Receipt Date | | Lot # | ceived | Article ID #'s |
| EpiPen ® | Feb. 5, | 2010 | NA | 3 | 2010-001-EP-1 |
| | | | | | thru |
| | | | | | 2010-001-EP-3 |
| EpiPen ® | Feb. 16, | 2010 | NA | 3 | 2010-001-EP-4 |
| | | | | | thru |
| | | | | | 2010-001-EP-6 |
| EpiPen ® | Feb. 25, | 2010 | 8GM782 | 24 | 2010-001-EP-7 |
| | | | | | thru |
| | | | | | 2010-001-EP-30 |
| EpiPen ® Jr | Mar. 15, | 2010 | NA | 12 | 2010-001-EPJ-1 |
| | | | | | thru |
| | | | | | 2010-001-EP-12 |
| EpiPen ® | Jul. 22, | 2010 | XX | 6 | 2010-02-EP-1 |
| | | | | | thru |
| | | | | | 2010-02-EP-6 |

Test and control articles were stored within the Division of Comparative Medicine (Rm. G05A3) at room temperature. Unused test and control article samples (per auto-injector type) are archived at Meridian Medical Technologies, Inc., 6350 Stevens Forest Rd., Columbia, Md., 21046.

3.2.5 Auto-injector Assignments (Groups P1, P2 and P3)

Auto-injector devices and pig study numbers for Groups P1, P2 and P3 are shown in Tables 7, 8 and 9 below.

TABLE 7
Animal Study Numbers and Auto-injector Devices (Group P1)

| | Test Article | Control Article |
|---|---|---|
| | (Anapen ® 300) | (EpiPen ®) |
| Pig Study Number | Gauge/Needle Size (″) | Gauge/Needle Size (″) |
| 2010-001-105-P1-D | 27 ga × 0.3″ | 22 ga × 0.6″ |
| 2010-001-106-P1-ND | 27 ga × 0.3″ | 22 ga × 0.6″ |
| 2010-001-107-P1-D | 27 ga × 0.3″ | 22 ga × 0.6″ |
| 2010-001-108-P1-ND | 27 ga × 0.3″ | 22 ga × 0.6″ |

TABLE 8
Animal Study Numbers and Auto-injector Devices (Group P2)

| | Test Article | Control Article |
|---|---|---|
| | (Twinject ® 0.15 mL) | (EpiPen ® Jr) |
| Pig Study Number | Gauge/Needle Size (″) | Gauge/Needle Size (″) |
| 2010-001-120-P2-D | 25 ga × 0.5″ | 22 ga × 0.5″ |
| 2010-001-121-P2-ND | 25 ga × 0.5″ | 22 ga × 0.5″ |
| 2010-001-122-P2-ND | 25 ga × 0.5″ | 22 ga × 0.5″ |
| 2010-001-123-P2-D | 25 ga × 0.5″ | 22 ga × 0.5″ |

TABLE 9
Animal Study Numbers and Auto-injector Devices (Group P3)

| | Test Article | Control Article |
|---|---|---|
| | (Twinject ® 0.30 mL) | (EpiPen ®) |
| Pig Study Number | Gauge/Needle Size (″) | Gauge/Needle Size (″) |
| 2010-02-229-P3-D | 25 ga × 0.5″ | 22 ga × 0.6″ |
| 2010-02-230-P3-ND | 25 ga × 0.5″ | 22 ga × 0.6″ |
| 2010-02-231-P3-ND | 25 ga × 0.5″ | 22 ga × 0.6″ |
| 2010-02-232-P3-D | 25 ga × 0.5″ | 22 ga × 0.6″ |

3.2.5 Injectate

All test and control article auto-injectors were supplied non-sterile and contained 0.75 ml water for injection mixed with 0.25 ml Omnipaque 300™ (Amersham Health Inc., Princeton, N.J.) per one ml of injectate. The injectate solution was mixed as a single batch and all test and control auto-injectors were filled from this single batch. The Anapen® 300 auto-injectors delivered 0.3 mL, the Twinject® 0.15 mL auto-injectors delivered 0.15 ml and the Twinject® 0.30 mL auto-injectors delivered 0.30 ml. Both control article auto-injectors (EpiPen® and EpiPen® Jr) delivered 0.3 mL.

The sponsor (Meridian Medical Technologies, Inc.) pre-filled all auto-injectors with non-sterile solution through the use of test protocol #R01-664 prior to shipping to the testing facility.

3.3 Equipment
Siemens Somatom Emotion 16 Scanner (serial #32407)
Engler A.D.S. 1000 Anesthesia Delivery System
Ohmeda Isoflurane Vaporizer
SurgiVet Pulse Oximeter
VWR International Traceable Digital Calipers
Fisher Scientific Traceable Extra Loud Timers
Cardinal Detecto Scale—Model VET-400
Dickson Chart Recorder—TH603
Analyze© 7.0 Software Suite

Equipment used in the study was in good working condition and was calibrated to the extent possible.

3.4 Pre-Study Procedural Assessment

One pre-study pig (ear tag #22) was sedated with Telazol (6 mg/kg, IM) and euthanized with Euthasol™ (10 mL/kg, IV) on the day after arrival. This animal was used to determine the specific location for study injection sites and the optimal size/placement/attachment method of denim patches onto the skin of the pig's thigh.

On the pre-assessment day, the cadaver pig was placed in dorsal recumbency within the study restraint device (V-trough) after euthanasia to assess injection site location. Due to presence of thigh skin folds, it was determined that the intramuscular (IM) injections should be administered lateral to, instead of vertically from, the top of the patella. The optimal size of the denim patch required for use in the main study was assessed. A rectangular piece of pre-cut denim (3″H×4″ W, 0.87 mm thick) was determined to be of sufficient size to cover the site of injection, with the top of the denim patch placed in line with the skin fold. Stapling the denim patch to the skin was found to be effective in holding the patch firmly in place. The patch was stapled on all four corners and then once in-between the staples, along the edge of the patch. Vet personnel were trained on the use of live test and control auto-injectors. Each operator held the pig's leg with the non-auto-injector hand for limb stabilization during injection.

3.5 Injection and CT Imaging Procedures

For the main study, animals were weighed and anesthetized in the Division of Comparative Medicine. Anesthesia was induced by the administration of Telazol (6 mg/kg, IM) and atropine (0.5 mg/kg, SQ). An ear vein catheter was placed. The animals were intubated and placed on isoflurane (1-3%) gas anesthesia. The anesthetized animals were placed on a transport cart, covered and transported to the Department of Radiology located in the Georgetown University Hospital (CT Suite #1). The depth of anesthesia was monitored by measuring heart rate, respiratory rate and pulse oximetry (SPO₂) throughout the procedure.

The CT suite was equipped with a Siemens Somatom Emotion 16 CT Scanner. The anesthetized pig was placed on the CT table, in a V-trough, with the head towards the front of the gantry. The anesthesia equipment was connected from behind the CT gantry.

In 50% of the animals, a 3″W×4″L pre-cut denim patch (Wrangler Hero®, regular fit) was stapled (Ethicon Endo-Surgery, 1-Proximate®, Skin Stapler (35 wide)) onto both thigh muscles lateral to the patella. The injection sites (one per thigh) were then measured using digital calipers (3 cm laterally from the top of the patella). The injection site was marked with indelible ink, either directly on the skin or on the denim patch.

Both the right and left thighs were injected simultaneously, with different technicians performing each injection. One technician used a control article auto-injector in the left thigh and the other technician used a test article auto-injector in the right thigh. The control or test article was placed on the marked site on the belly of the designated muscle. Both auto-injectors were positioned at an approximate 90 degree angle on the muscle belly.

At the time of the injections, the study director provided a count down [5, 4, 3, 2 & 1] to announce the beginning of the simultaneous injections and started the study timers. After injection, both the control and test article auto-injectors were held in place for five (5) seconds (reduced from labeled 10 seconds) to assure delivery of drug and technicians immediately left the room so that the first CT image (0 time point) could be expedited. Across all test and control articles, after the auto-injectors were removed from the muscle at the five (5) second point, all injectate appeared to have been fully dispensed. After the injection and CT imaging were completed, the auto-injectors were placed in a plastic tray for CT scanning of post-injection needle length. The EpiPen® and EpiPen® Jr scans were conducted through the orange needle sharps cover.

All test and control articles were placed in a sharps container following measurement of post-injection needle lengths by CT scans.

CT volumes were obtained at 0, 1, 2, 3, 4, 5, 7, 9, 11, 13 and 15 minutes. All of the images were saved in DICOM format for subsequent analysis. CT scanning began as soon as all personnel left the CT room.

3.6 Test and Control Article Administration

Test and control articles were administered to animals in Groups P1 and P2 as shown below (Table 10).

TABLE 10
Test and Control Article Administration by Group, Study Date, Pig
Study Number and Test and Control Article Number

| | Study | | Test Article Number | Control Article Number |
|---|---|---|---|---|
| Group | Date | Pig Study Number | (Anapen ® 300) | (EpiPen ®) |
| P1 | Mar. 6, 2010 | 2010-001-105-P1-D | 2010-001-AN-8 | 2010-001-EP-6 |
| P1 | Mar. 6, 2010 | 2010-001-106-P1-ND | 2010-001-AN-9 | 2010-001-EP-7 |
| P1 | Mar. 6, 2010 | 2010-001-107-P1-D | 2010-001-AN-10 | 2010-001-EP-8 |
| P1 | Mar. 6, 2010 | 2010-001-108-P1-ND | 2010-001-AN-11 | 2010-001-EP-9 |
| | Study | | Test Article Number | Control Article Number |
| Group | Date | Pig Study Number | (Twinject ® 0.15 mL) | (EpiPen ® Jr) |
| P2 | Mar. 20, 2010 | 2010-001-120-P2-D | 2010-001-TWJ-3 | 2010-001-EPJ-3 |
| P2 | Mar. 20, 2010 | 2010-001-121-P2-ND | 2010-001-TWJ-1 | 2010-001-EPJ-1 |
| P2 | Mar. 20, 2010 | 2010-001-122-P2-ND | 2010-001-TWJ-2 | 2010-001-EPJ-2 |
| P2 | Mar. 20, 2010 | 2010-001-123-P2-D | 2010-001-TWJ-4 | 2010-001-EPJ-4 |
| | Study | | Test Article Number | Control Article Number |
| Group | Date | Pig Study Number | (Twinject ® 0.30 mL) | (EpiPen ® Jr) |
| P3 | Jul. 24, 2010 | 2010-02-229-P3-ND | 2010-02-TW-1 | 2010-02-EP-1 |
| P3 | Jul. 24, 2010 | 2010-02-230-P3-D | 2010-02-TW-3 | 2010-02-EP-3 |
| P3 | Jul. 24, 2010 | 2010-02-231-P3-D | 2010-02-TW-4 | 2010-02-EP-4 |
| P3 | Jul. 24, 2010 | 2010-02-232-P3-ND | 2010-02-TW-2 | 2010-02-EP-2 |

3.7 Image Acquisition Protocol

The CT images were acquired at 110 kV with a rotation time of 0.1 seconds. Narrow collimation was used with a slice width of 1.0 mm and 1.0 mm collimation using the B30s medium smooth reconstruction kernel. The Window settings were “ABDOMEN” with a reconstruction increment of 1.0 mm.

The steps for each pig were as follows:

1. Obtain an initial tomogram (scout) image (FIG. 62)

2. Define a region of interest for all subsequent sequences

3. Left and right thigh auto-injections were acquired simultaneously

4. The set of CT scans were taken for 15 minutes

5. Image reconstruction was performed after completion of step 4

6. Images were burned and archived to CD

All images were archived on CD using the DICOM medical image file format. This is a standard format in medical imaging which contains both the images and a header file with complete information about the image acquisition, including the imaging modality and time of acquisition.

3.8 Image Analysis

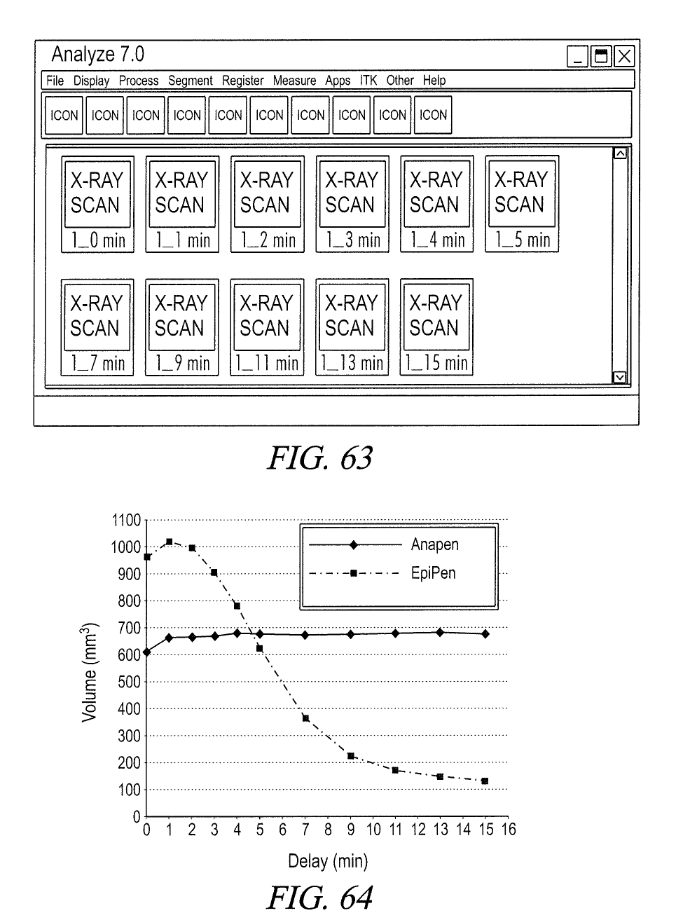

The purpose of the image analysis was to determine the spread of injectate. The CT image analysis was done using the Analyze© 7.0 Software Suite from the Mayo Clinic (http://www.analyzedirect.com) shown in FIG. 63. This tool allows the interactive segmentation of CT volumes using the well-established techniques of thresholding and region growing. The maximum threshold was selected to be greater than the maximum value of the image range. The minimum threshold was selected to be larger than the tissue intensities surrounding the injection region. It should be noted that on CT this provides a 3D segmentation, from which a volume can be computed as the sum of all voxels within a series of axial scans.

A brief overview of the steps used to compute the injectate volume at each time interval is summarized below:
Sort datasets from each study into volumes
Sub-volume of interest is identified and saved
A minimum threshold value is chosen such that boundaries of injectate site are clearly demarcated (this threshold is subsequently used for all studies)
Two objects are defined to denote left and right injection sites
Using seed points and a region growing algorithm, both left and right injection sites are computed and saved as objects
Volume measurement tools operate on the saved objects to generate statistics of injectate spread
If multiple injectate pools exist, at least one seed is defined for each injectate pool and region growing algorithm is applied to all the seeds for each injection site
Volume measurement tools operate on the saved objects to generate statistics of injectate dispersion

3.9 Euthanasia

Immediately after CT scanning was completed, pigs were euthanized using a commercially available euthanasia solution (Euthasol™) by giving a minimum of 1 mL/10 lbs body weight, IV. Pig carcasses were returned to the necropsy room in the Division of Comparative Medicine. The skin directly over the injection site was incised with a scalpel, and the depth of the combined skin/fat layer was measured using digital calipers.

### 4.0 Results

4.1 Animal Acclimation and Observations

Thirteen (13) female Yorkshire pigs were purchased from Thomas D. Morris, Inc. (Reisterstown, MD) and arrived at Georgetown University, Division of Comparative Medicine on Feb. 25, 2010; Mar. 3, 2010; Mar. 17, 2010 and Jul. 21, 2010.

On arrival, all animals were either gang or individually housed and received feed and water, per protocol. The animal room environmental temperatures were targeted between 68°—81° F. The actual room temperatures varied between 68°−75° F. The animal room relative humidity was targeted between 30-70% humidity. The actual room relative humidity varied between 32-62%.

The pre-study animal (ear tag #22) was euthanized the day after arrival. The remaining animals were acclimated a minimum of three (3) days. All twelve (12) study animals were examined prior to study initiation and were determined to be suitable for study. All animals were within the required weight range for study. CT scanning was performed after bilateral intramuscular injections were administered using control article (EpiPen® and EpiPen® Jr) and test article (Anapen® 300, Twinject® 0.30 mL and Twinject® 0.15 mL) auto-injectors, respectively. Animals were euthanized immediately after scanning was completed.

Acclimation Period:

During the acclimation period, minor clinical conditions were observed in two (2) pigs (Table 11). No pigs required clinical treatment. All pigs were bright, alert and responsive (BAR).

TABLE 11
Animal Observations During Study Acclimation

| | | | | Date |
|---|---|---|---|---|
| | Condition | Date | Treat- | Condition |
| Animal # | Observed | Observed | ment | Resolved |
| 2010-001-107-P1-D | Irritation on | Mar. 4, | None | Mar. 5, 2010 |
| | tip of nose | 2010 | required | (end of |
| | | | | study) |
| 2010-001-121-P2-ND | Scratch on | Mar. 18, | None | Mar. 20, 2010 |
| | nose | 2010 | required | (end of |
| | | | | study) |

4.2 CT Scan Analysis

CT scan calculations using the Analyze© 7.0 Software Suite are done on a per voxel basis. Therefore, in addition to a volume measure (in mm³) for each time interval, the mean and standard deviation of voxel intensities within the segmented object denoting each injection site is provided. The volume measure is in direct correlation to the dispersion and uptake of the injectate within tissue. The mean and standard deviation of voxel intensities together provide a view of the spread of the injectate contrast agent (Omnipaque™) within the injection site and its relative uptake within tissue.

The results for all studies are presented within this section. The results are broken down into two sections, one for each animal study group (P1, P2 and P3).

4.2.1 Study Group P1 (EpiPen® vs. Anapen® 300)

As an example for Group P1 results, the segmentation values using Analyze© 7.0 Software Suite are summarized in Table 12 (below) for pig #106. The table on the left summarizes results for the control article (EpiPen®) auto-injection site in the left thigh and the table on the right summarizes results for the test article (Anapen® 300) auto-injection site in the right thigh. A plot of remaining volume over time for pig #106 is shown in FIG. 64 for both the test article (Anapen® 300) and the control article (EpiPen®).

TABLE 12
Summary of Voxel Intensities and Volume Measure of Injectate
Uptake - Control and Test Article Objects - Group P1 (Pig # 106)

| Control Article: | | | | Test Article: | | | |
|---|---|---|---|---|---|---|---|
| EpiPen ® Auto-injector | | | | Anapen ® 300 Auto-injector | | | |
| (left thigh) | | | | (right thigh) | | | |
| | Voxel | | | | Voxel | | |
| Delay | Intensities | | Volume | Delay | Intensities | | Volume |
| (min) | Mean | Std. dev | (mm³) | (min) | Mean | Std. dev | (mm³) |
| 0 | 523.64 | 175.81 | 958.36 | 0 | 721.72 | 351.08 | 613.36 |
| 1 | 467.00 | 166.15 | 1018.30 | 1 | 664.37 | 312.82 | 664.05 |
| 2 | 442.96 | 163.97 | 991.95 | 2 | 653.52 | 315.12 | 661.84 |
| 3 | 414.46 | 159.72 | 905.45 | 3 | 648.38 | 314.40 | 668.27 |
| 4 | 399.76 | 164.03 | 779.52 | 4 | 640.61 | 314.45 | 679.54 |
| 5 | 390.78 | 172.00 | 621.80 | 5 | 638.01 | 313.70 | 678.13 |
| 7 | 407.89 | 194.70 | 366.73 | 7 | 630.63 | 317.57 | 673.50 |
| 9 | 442.75 | 209.02 | 224.90 | 9 | 628.24 | 316.24 | 675.31 |
| 11 | 461.69 | 197.06 | 167.77 | 11 | 622.34 | 314.39 | 678.33 |
| 13 | 452.55 | 179.14 | 145.04 | 13 | 617.86 | 314.31 | 682.56 |
| 15 | 441.33 | 167.58 | 129.35 | 15 | 615.10 | 312.57 | 675.31 |

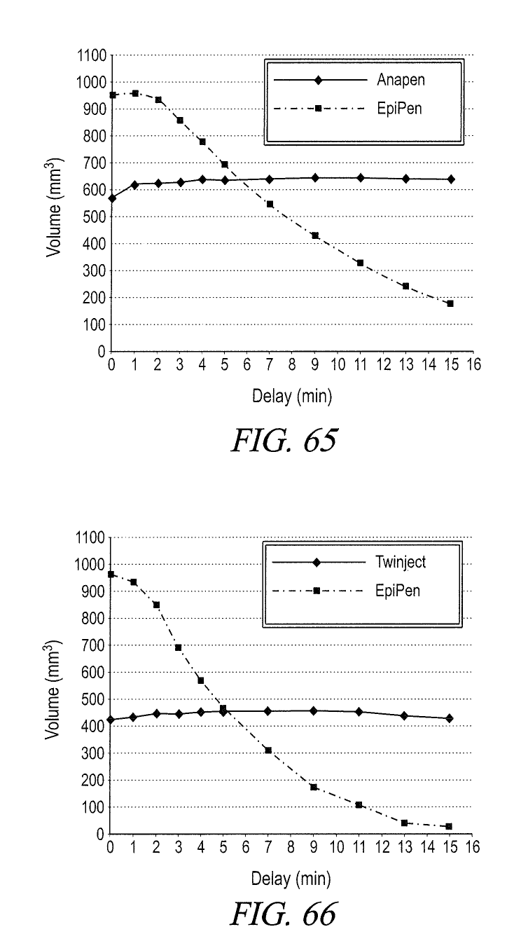

A summary of average volume of injectate uptake for all study group P1 tests (Pig #105-108) is provided in Table 13 (below), and a plot of the injectate uptake over all scans is provided in FIG. 65.

TABLE 13
Average Volume Measures of Injectate Uptake - Control
(EpiPen ®) and Test (Anapen ®300)
Articles - Group P1 (Pig # 105-108)

| | Delay | EpiPen ® | Anapen ® 300 |
|---|---|---|---|
| | (min) | Volume (mm³) | Volume (mm³) |
| Aggregate | 0 | 949.76 | 576.70 |
| Summary | 1 | 955.84 | 614.11 |
| | 2 | 928.53 | 622.11 |
| | 3 | 856.06 | 629.75 |
| | 4 | 775.19 | 633.57 |
| | 5 | 692.31 | 638.70 |
| | 7 | 543.96 | 638.95 |
| | 9 | 426.57 | 643.98 |
| | 11 | 324.43 | 643.03 |
| | 13 | 241.25 | 643.33 |
| | 15 | 175.47 | 640.01 |

In all study group P1 trials, the EpiPen® injectate reached peak volume within the first minute and decreased to 20%, by volume, by the end of the study. No appreciable decrease in Anapen® injectate volume was noticed by the end of image acquisition for the four P1 trials.

4.2.2 Study Group P2 (EpiPen® Jr vs. Twinject® 0.15 mL)

Comparison of the EpiPen® Jr (control article) with the Twinject® 0.15 mL (test article) for pig #120 in the P2 study group is summarized in Table 14 below. As with the P1 group, the table on the left summarizes results for the control article (EpiPen® Jr) injection site in the left thigh, and the table on the right summarizes results for the test article (Twinject® 0.15 mL) injection site in the right thigh. A plot of injectate volume uptake over time for Pig #120 is shown in FIG. 66 for both the test article (Twinject® 0.15 mL) and the control article (EpiPen® Jr).

TABLE 14
Summary of Voxel Intensities and Volume Measure of lnjectate
Uptake - Control and Test Article Objects - Group P2 (Pig #120)

| Control Article: | | | | Test Article: | | | |
|---|---|---|---|---|---|---|---|
| EpiPen ® Jr (left thigh) | | | | Twinject ® 0.15 mL (right thigh) | | | |
| | Voxel | | | | Voxel | | |
| Delay | Intensities | | Volume | Delay | Intensities | | Volume |
| (min) | Mean | Std. dev | (mm³) | (min) | Mean | Std. dev | (mm³) |
| 0 | 433.69 | 121.83 | 970.83 | 0 | 666.63 | 328.79 | 424.46 |
| 1 | 403.34 | 106.69 | 935.22 | 1 | 635.31 | 311.47 | 434.92 |
| 2 | 380.69 | 97.97 | 850.73 | 2 | 614.46 | 298.84 | 445.18 |
| 3 | 368.63 | 92.96 | 691.01 | 3 | 610.17 | 304.75 | 445.38 |
| 4 | 361.50 | 86.23 | 569.10 | 4 | 597.58 | 299.85 | 452.22 |
| 5 | 351.75 | 76.92 | 466.71 | 5 | 596.15 | 300.89 | 450.81 |
| 7 | 333.88 | 63.79 | 311.81 | 7 | 585.18 | 301.15 | 453.03 |
| 9 | 324.20 | 56.59 | 174.41 | 9 | 576.11 | 296.36 | 454.84 |
| 11 | 309.90 | 44.07 | 105.61 | 11 | 564.71 | 290.63 | 452.42 |
| 13 | 294.99 | 27.95 | 42.04 | 13 | 562.37 | 292.15 | 436.13 |
| 15 | 290.46 | 28.20 | 25.35 | 15 | 559.28 | 291.86 | 427.48 |

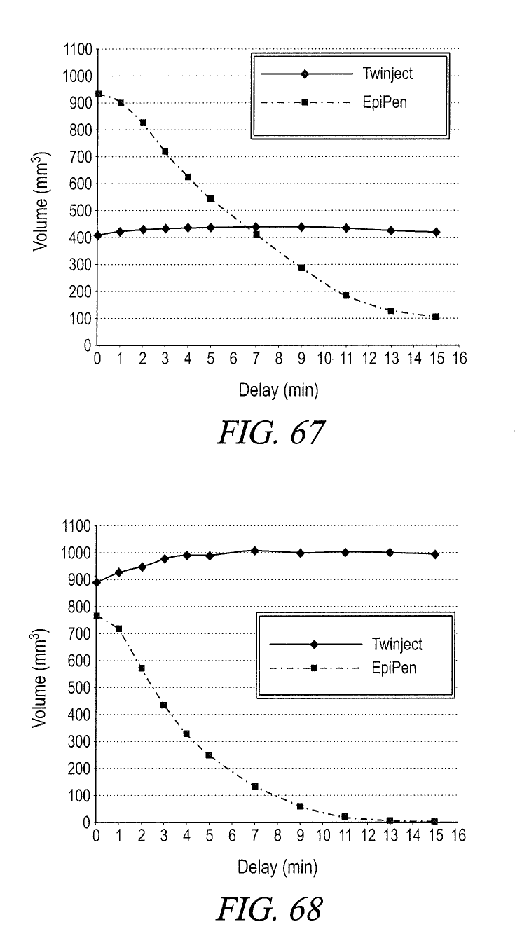

A summary of average volume of injectate uptake for all study group P2 tests (pig #120-123) is provided in Table 15 (below), and a plot of the injectate volume uptake over all scans is provided in FIG. 67.

TABLE 15
Average Volume Measures of Injectate Uptake -
Control (EpiPen ® Jr and Test (Twinject ® 0.15
mL) Articles - Group P2 (Pig # 120-123)

| | Delay | EpiPen ® Jr | Twinject ® 0.15 mL |
|---|---|---|---|
| | (min) | Volume (mm³) | Volume (mm³) |
| Aggregate | 0 | 934.77 | 412.04 |
| Summary | 1 | 901.12 | 424.36 |
| | 2 | 827.85 | 432.66 |
| | 3 | 721.23 | 436.48 |
| | 4 | 628.04 | 436.63 |
| | 5 | 546.87 | 439.90 |
| | 7 | 413.40 | 442.32 |
| | 9 | 287.87 | 439.65 |
| | 11 | 188.90 | 436.78 |
| | 13 | 132.07 | 428.23 |
| | 15 | 107.63 | 422.35 |

In all study group P2 trials, the EpiPen® Jr injectate reached peak dispersion volume within the first minute and decreased to 12%, by volume, by the end of the study. No appreciable decrease of Twinject® 0.15 mL injectate volume was noticed by the end of image acquisition for the four P2 trials.

4.2.3 Study Group P3 (EpiPen® vs. Twinject® 0.30 mL)

Comparison of the EpiPen® (control article) with the Twinject® 0.30 mL (test article) for pig #229 in the P3 study group is summarized in Table 16 below. As with the P1 group, the table on the left summarizes results for the control article (EpiPen®) injection site in the left thigh and the table on the right summarizes results for the test article (Twinject® 0.30 mL) injection site in the right thigh. A plot of injectate volume uptake over time for Pig #229 is shown in FIG. 68 for both the test article (Twinject® 0.30 mL) and the control article (EpiPen®).

TABLE 16
Summary of Voxel Intensities and Volume Measure of Injectate
Uptake - Control and Test Article Objects - Group P3 (Pig #229)

| Control Article: | | | | Test Article: | | | |
|---|---|---|---|---|---|---|---|
| EpiPen ® Auto-injector | | | | Twinject ® 0.30 mL Auto-injector | | | |
| (left thigh) | | | | (right thigh) | | | |
| | Voxel | | | | Voxel | | |
| Delay | Intensities | | Volume | Delay | Intensities | | Volume |
| (min) | Mean | Std. dev | (mm³) | (min) | Mean | Std. dev | (mm³) |
| 0 | 452.74 | 155.29 | 765.04 | 0 | 589.89 | 237.71 | 885.33 |
| 1 | 404.15 | 120.89 | 719.37 | 1 | 543.98 | 203.24 | 927.98 |
| 2 | 378.13 | 111.08 | 570.31 | 2 | 521.32 | 188.56 | 947.09 |
| 3 | 364.24 | 95.92 | 436.93 | 3 | 509.43 | 180.98 | 977.47 |
| 4 | 346.26 | 83.73 | 325.49 | 4 | 499.74 | 176.05 | 991.35 |
| 5 | 337.65 | 78.84 | 246.43 | 5 | 493.79 | 170.22 | 991.15 |
| 7 | 319.08 | 58.31 | 131.16 | 7 | 480.37 | 162.41 | 1008.45 |
| 9 | 306.40 | 49.16 | 56.33 | 9 | 468.47 | 154.49 | 998.19 |
| 11 | 292.94 | 31.87 | 16.69 | 11 | 463.20 | 151.24 | 1003.22 |
| 13 | 274.80 | 16.00 | 3.02 | 13 | 455.44 | 147.29 | 1000.00 |
| 15 | 0.00 | 0.00 | 0.00 | 15 | 449.63 | 141.50 | 992.96 |

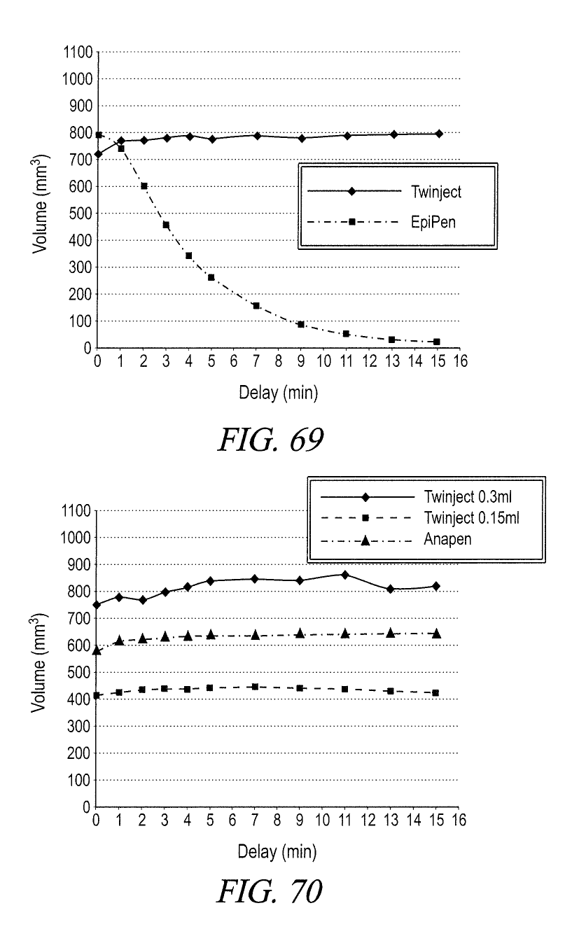

A summary of average volume of injectate uptake for study group P3 tests (pig #229-232) is provided in Table 17 (below) and a plot of the injectate volume uptake over all scans is provided in FIG. 69.

TABLE 17
Average Volume Measures of Injectate Uptake -
Control (EpiPen ® and Test (Twinject ® 0.30
mL) Articles - Group P3 (Pig # 229-232)

| | Delay | EpiPen ® | Twinject ® 0.30 mL |
|---|---|---|---|
| | (min) | Volume (mm³) | Volume (mm³) |
| Aggregate | 0 | 791.94 | 721.18 |
| Summary | 1 | 740.95 | 768.86 |
| | 2 | 599.53 | 771.92 |
| | 3 | 454.13 | 780.38 |
| | 4 | 341.73 | 788.62 |
| | 5 | 260.16 | 777.91 |
| | 7 | 156.06 | 788.47 |
| | 9 | 85.25 | 779.47 |
| | 11 | 50.74 | 788.52 |
| | 13 | 30.08 | 792.14 |
| | 15 | 21.78 | 794.91 |

In study group P3 trials, the EpiPen® injectate reached peak volume immediately following injection and decreased to less than 3%, by volume, by the end of the study. No appreciable decrease of Twinject® 0.3 mL injectate volume was noticed by the end of image acquisition for all animals, with the exception of pig #230. In animal #230, the Twinject® 0.3 mL injectate was deployed close to the bone. For this reason, the injectate could not be delineated from the bone automatically. Therefore, the test article injectate site had to be manually segmented for this one study. The test article injectate dispersion for this test is slightly different from the other three tests.

4.2.4 Comparison of Test Article Injectate Dispersion and Uptake

Comparison of the three test articles, on average, showed the Twinject® 0.30 mL occupied a larger injectate spread volume in situ, but reached peak volume more slowly, than either the Twinject® 0.15 mL or the Anapen® 300. The Twinject® 0.15 mL auto-injector dispensed 50% of the volume of the Twinject® 0.30 mL and the Anapen® 300. None of the test articles displayed appreciable uptake of injectate once injected, either in general or relative to one another. This comparison is shown in FIG. 70.

4.2.5 Comparison of Control Article Injectate Dispersion and Uptake

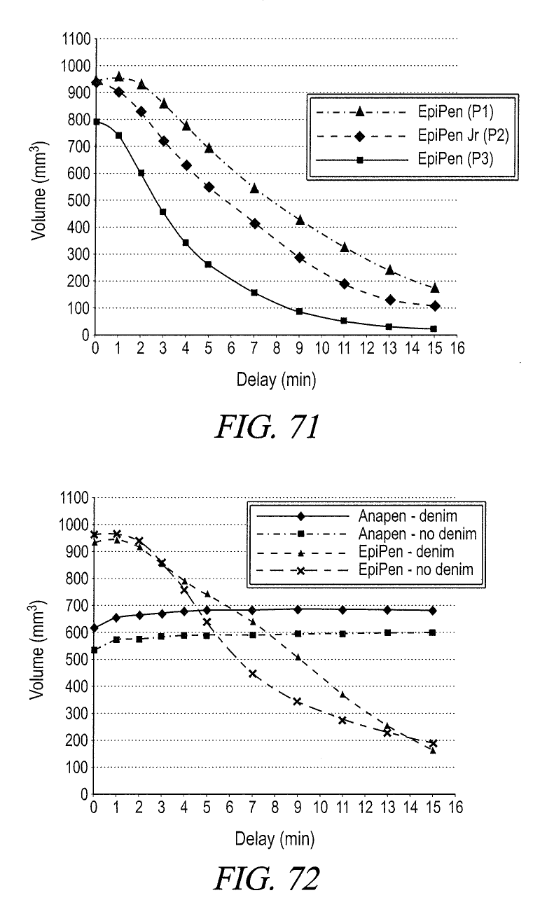

Comparison of the two control articles, on average, showed the EpiPen® and EpiPen® Jr injectate dispersion volumes to be similar. The average peak dispersion volume measurement for EpiPen® (Group P1 and Group P3) and for EpiPen® Jr (Group P2) was 1, 0 and 0 min, respectively. Injectate uptake was also similar (80%, 97% and 88%, respectively). This comparison is shown in FIG. 71.

4.2.6 Denim Patch vs. Direct Skin Injections

A total of 24 auto-injections were used in this composite study. Twelve (12) injections were given through denim material and twelve (12) were given directly through the skin. Group P1 (Anapen® 300 and EpiPen®), Group P2 (Twinject® 0.15 mL and EpiPen® Jr) and Group 3 (Twinject® 0.30 mL and EpiPen®) each had four (4) injections—two (2) through denim and two (2) directly through skin. The denim thickness was 0.87 mm, and the average skin/fat layer was 2.3 mm (Table 19). There were no appreciable differences in either injectate dispersion or uptake in either group with respect to whether the article was applied through denim or skin. This is shown in FIGS. 72-74.

FIG. 72 is a comparison of denim patch vs. direct skin auto-injections—Anapen® 300 and EpiPen® (Group 1—Pig #105-108).

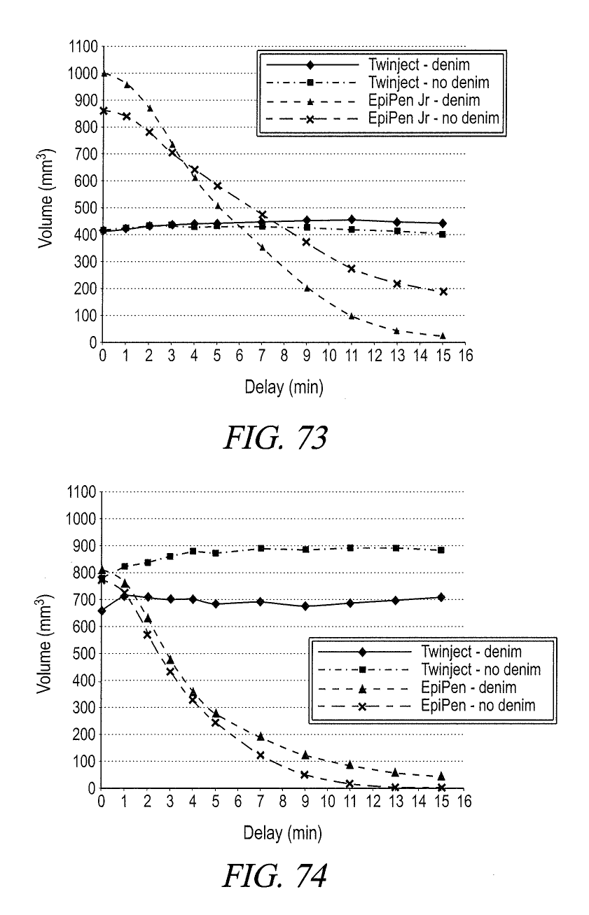

FIG. 73 is a comparison of denim patch vs. direct skin auto-injections—Group P2 Twinject® 0.15 mL and EpiPen® Jr (Pig #120-123).

FIG. 74 is a comparison of denim patch vs. direct skin auto-injections—Group P3 Twinject® 0.30 mL and EpiPen® (Pig #229-232).

4.2.7 Post-Injection Needle Lengths

Test and control article auto-injections were performed on Mar. 6, 2010, Mar. 20, 2010 and Jul. 24, 2010. All needles were held intramuscularly for five (5) seconds after injection. After removal, auto-injectors were scanned by CT to measure needle length within 20-37 minutes after the initial animal scan.

Post-injection needle lengths were measured using Analyze© 7.0 Software Suites. The post-injection needle scans were loaded into Analyze©, and threshold was adjusted such that only needle and plastic housing were visible. The ‘Line—Measure’ tool was used to define start and end points of the distance measure. For each case of test and control article, the tip of the needle was chosen as the start point, and the base of needle proximal to the plastic housing was chosen as the end point. Three measures were taken of each needle (in inches).

Summary of labeled (pre-injection) vs. measured (post-injection) needle lengths is shown in Table 18.

TABLE 18
CT Scan Measurements of Test and Control Article Needle Length
(″) Post Auto-Injection vs. Labeled Needle Length (″)

| | | Test Article (Anapen ® 300) | | Control Article (EpiPen ®) | |
|---|---|---|---|---|---|
| Study | | Needle Length (″) | | Needle Length (″) | |
| Group | Animal Study # | Labeled vs. Measured | | Labeled vs. Measured | |
| P1 | 2010-001-105-P1-D | 0.30″ | 0.33″ | 0.60″ | 0.61″ |
| | 2010-001-106-P1-ND | 0.30″ | 0.30″ | 0.60″ | 0.62″ |
| | 2010-001-107-P1-D | 0.30″ | 0.29″ | 0.60″ | 0.59″ |
| | 2010-001-108-P1-ND | 0.30″ | 0.32″ | 0.60″ | 0.60″ |
| | | Test Article (Twinject ® 0.15 mL) | | Control Article (EpiPen ® Jr) | |
| | | Needle Length (″) | | Needle Length (″) | |
| | | Labeled vs. Measured | | Labeled vs. Measured | |
| P2 | 2010-001-120-P2-D | 0.50″ | 0.52″ | 0.50″ | 0.57″ |
| | 2010-001-121-P2-ND | 0.50″ | 0.50″ | 0.50″ | 0.58″ |
| | 2010-001-122-P2-ND | 0.50″ | 0.48″ | 0.50″ | 0.57″ |
| | 2010-001-123-P2-D | 0.50″ | 0.47″ | 0.50″ | 0.56″ |
| | | Test Article (Twinject ® 0.30 mL) | | Control Article (EpiPen ®) | |
| | | Needle Length (″) | | Needle Length (″) | |
| | | Labeled vs. Measured | | Labeled vs. Measured | |
| P3 | 2010-02-229-P3-ND | 0.50″ | 0.50″ | 0.60″ | 0.59″ |
| | 2010-02-230-P3-D | 0.50″ | 0.47″ | 0.60″ | 0.61″ |
| | 2010-02-231-P3-D | 0.50″ | 0.48″ | 0.60″ | 0.60″ |
| | 2010-02-232-P3-ND | 0.50″ | 0.49″ | 0.60″ | 0.59″ |

Test Articles:

Anapen® 300 (group P1), Twinject® 0.15 mL (group P2) and Twinject® 0.30 mL (group P3) needle lengths measured within ±0.03″ of the labeled lengths.

Control Articles:

EpiPen® (group P1 and group 3)—needle lengths measured within ±0.02″ of the labeled lengths. EpiPen® Jr (group P2) needle lengths measured slightly higher than as labeled, with a maximal difference of 0.08″.

4.3 Skin/Fat Layer Measurements at Auto-Injection Sites

The combined depth (mm) of the skin/fat layer directly over the auto-injection site was measured by digital calipers, post mortem (Table 19).

TABLE 19
Injection Site Skin/Fat Layer Measurements

| | | Measured Skin/Fat Depth | |
|---|---|---|---|
| | Day of Study | (mm) | |
| Animal Study # | Weight (kg) | Left Thigh | Right Thigh |
| 2010-001-105-P1-D | 32.4 | 1.86 | 2.41 |
| 2010-001-106-P1-ND | 30.6 | 1.65 | 2.04 |
| 2010-001-107-P1-D | 30.6 | 1.80 | 1.91 |
| 2010-001-108-P1-ND | 30.6 | 2.64 | 2.38 |
| 2010-001-120-P2-D | 35.6 | 2.01 | 1.93 |
| 2010-001-121-P2-ND | 36.9 | 2.18 | 2.51 |
| 2010-001-122-P2-ND | 33.0 | 2.24 | 2.22 |
| 2010-001-123-P2-D | 38.1 | 3.57 | 2.37 |
| 2010-02-229-P3-ND | 35.6 | 1.91 | 3.27 |
| 2010-02-230-P3-D | 32.6 | 2.09 | 2.00 |
| 2010-02-231-P3-D | 32.4 | 1.93 | 2.28 |
| 2010-02-232-P3-ND | 34.0 | 2.26 | 2.35 |

The average measure of the skin fat/layer of the left thigh vs. the right thigh in all animals was 2.18 mm vs. 2.30 mm, respectively, with an average depth of 2.24 mm.

4.4 Record Retention

All study data, including but not limited to animal data, body weights, food consumption, physical examinations, study protocol and any communications concerning the conduct of the study is archived with Meridian Medical Technologies, Inc., 6350 Stevens Forest Road, Columbia, Md. 21046. Unused test and control articles, and any additional study data generated by the sponsor, are archived with Meridian Medical Technologies, Inc. at the address listed above.

### 5.0 Discussion and Conclusions

5.1 CT Scan Analysis—Study Group P1 (EpiPen® vs. Anapen® 300)

In study group P1 trials, the average EpiPen® injectate dispersion volume immediately following injection was 949.76 mm³ vs. the Anapen® 300 measured volume of 576.70 mm³. The EpiPen® injectate reached peak measured dispersion volume within one (1) minute, with average injectate uptake of 80%, by volume, at the 15 minute time point. In contrast, the Anapen® 300 injectate reached peak dispersion volume in most trials within the first nine (9) minutes, and in most cases, uptake by volume was negligible at the 15 minute time point.

The larger average initial injectate dispersion volume and the greater injectate volume uptake seen 15 minutes post-injection (80% vs. negligible uptake) demonstrated that the EpiPen® auto-injector delivered injectate into muscle tissue with greater efficiency than the Anapen® 300 auto-injector in this study. As the injectate volumes of the two auto-injectors were identical (0.3 mL), it can be hypothesized that the greater delivery efficiency of the EpiPen® may be due to its larger needle size (22 ga. vs. 27 ga.), longer needle length (0.6″ vs. 0.3″) and/or greater spring force (23.0 lbs vs. 2.1 lbs). The larger bore needle may allow for wider dispersion at the needle tip, the longer needle deposits injectate deeper into the muscle tissue and the greater spring force pressure may drive the injectate into the tissue.

The only exception noted in study group P1 trials was the third pig study (pig #105), where the control article injectate site touched the bone. This was a marginal condition, and the results do not show appreciable change from the norm in injectate volume

5.2 CT Scan Analysis—Study Group P2 (EpiPen® Jr vs. Twinject® 0.15 mL)

In all study group P2 trials, the average EpiPen® Jr injectate dispersion volume immediately following auto-injection was 934.77 mm³ vs. the Twinject® 0.15 mL measured volume of 412.04 mm³. The EpiPen® Jr injectate reached peak measured dispersion volume within the first minute, with average injectate uptake of 88%, by volume, at the 15 minute time point. In contrast, the Twinject® 0.15 mL injectate reached peak dispersion volume in most trials within the first seven (7) minutes and, in most cases, uptake was negligible, by volume, by the end of study.

The more rapid injectate dispersion, the greater peak dispersion volume and the greater average uptake of the injectate from the site on injection (88% vs. negligible) demonstrated that the EpiPen® Jr delivered injectate into muscle tissue with greater efficiency than the Twinject® 0.15 mL in this study. The auto-injector needle lengths post-injection were similar; however, other parameters of the EpiPen® Jr and Twinject® 0.15 mL differed, such as: injectate volumes (0.3 mL vs. 0.15 mL), needle gauge (22 ga. vs. 25 ga.) and spring force (23.0 vs. 6.5 lbs), respectively. It is hypothesized that the greater delivery efficiency of EpiPen® Jr vs. Twinject® 0.15 mL may be attributed to the larger needle size of the EpiPen® Jr and greater spring force. It is also noteworthy that although the Twinject® 0.15 mL injectate volume was only 50% of the EpiPen® Jr injectate volume, it was delivered at the same approximate depth (0.5″), but uptake remained negligible at the 15 minute time point.

5.3 CT Scan Analysis—Study Group P3 (EpiPen″ vs. Twinject® 0.30 mL)

In study group P3 trials, the average EpiPen® injectate dispersion volume immediately following auto-injection was 791.94 mm³ vs. the Twinject® 0.30 mL measured volume of 721.18 mm³. The EpiPen® injectate reached peak measured dispersion volume within the zero (0) minute, with average injectate uptake of 97%, by volume, at the 15 minute time point. In contrast, the Twinject® 0.30 mL injectate reached peak dispersion volume in most trials between 7-15 minutes and, in most cases, uptake was negligible, by volume, by the end of study. In one trial (animal #230) the injection was deployed close to bone and the injectate could not be delineated from the bone automatically—the test article site was therefore manually segmented for this one study. The test article injectate for animal #230 reached a peak volume at the one (1) minute interval and then dispersed by 14% at the 15 minute interval. This difference in dispersion profile may be due to the physiology of the tissue surrounding the bone, as it was likely that some of the injectate seeped along the surface of the bone. This seepage would be difficult to detect manually using the Analyze© software and is a likely source of the difference in dispersion profile for this animal.

The more rapid injectate dispersion, the greater peak dispersion volume and greater average uptake of the injectate from the site on injection (97% vs. negligible) demonstrated that the EpiPen® delivered injectate into muscle tissue with greater efficiency than the Twinject® 0.30 mL in this study. The auto-injector injection volumes, and needle lengths post-injection, and were similar; however, other parameters of the EpiPen® and Twinject® 0.30 mL differed, such as: needle gauge (22 ga. vs. 25 ga.) and spring force (23.0 vs. 6.5 lbs), respectively. It is hypothesized that the greater delivery efficiency of EpiPen® vs. Twinject® 0.30 mL may be attributed to the larger needle size of the EpiPen® and greater spring force. It is also noteworthy that although the Twinject® 0.30 mL and EpiPen® injectate volumes were the same and were delivered at the similar depths (0.5″ vs. 0.6″), Twinject® 0.30 mL injectate uptake remained negligible at the 15 minute time point.

5.4 Comparison of Injectate Dispersion and Uptake—Test Articles (Anapen® 300, Twinject® 0.15 mL and Twinject® 0.30 mL)

Comparison of the three test articles showed the Twinject® 0.30 mL auto-injector demonstrated a larger initial injectate dispersion volume (721.18 mm³) vs. the Twinject® 0.15 mL (412.04 mm³) and the Anapen® 300 (576.70).The Anapen® 300 reached peak injectate dispersion volume (643.98 mm³) at nine (9) minutes vs. the Twinject® 0.15 mL (442.32 mm³) at seven (7) minutes and the Twinject® 0.30 mL (794.91 mm³) at fifteen (15) minutes. The spring force of the Anapen® 300 vs. the Twinject® 0.15 mL and Twinject® 0.30 mL was 2.1 lbs. vs. 6.5 lbs and 6.5 lbs, respectively.

On average the Twinject® 0.30 mL auto-injector occupied a larger injectate volume in situ (pooled) and a larger peak volume than either the Twinject® 0.15 mL or Anapen® 300 auto-injectors. This data suggests that the Twinject® 0.30 mL dispersed injectate more widely than the Anapen® 300 (at equal injection volumes) and had slower peak dispersion. These findings could be explained by the larger volume of the Twinject® 0.30 mL vs. the Twinject® 0.15 mL auto-injector and the larger needle gauge of the Twinject® 0.30 mL vs. the Anapen® 300 auto-injector. The Twinject® 0.15 mL, with 50% of the injection volume of the other test articles, reached peak dispersion more rapidly than either the Twinject® 0.30 mL or Anapen® 300 auto-injectors. The spring force of the Twinject® 0.15 mL (6.5 lbs) may have contributed to the more rapid peak injectate dispersion but the spring force was only slightly greater than either the Twinject® 0.30 mL or Anapen® 300 auto-injectors (2.1 to 6.5. lbs).

None of the three test articles displayed appreciable average uptake of injectate at the 15 minute time point, either in general or relative to one another, suggesting that neither test article delivered injectate in a manner that led to dissemination within the muscle tissue. Needle gauge, needle length and spring force may be contributing factors to the lack of injectate uptake.

5.5 Comparison of Injectate Dispersion and Uptake in Control Articles—EpiPen® (Groups P1, P3) vs. EpiPen® Jr (Group P2)

Comparison of the two control articles showed the EpiPen® auto-injector (group P1) and (group P3) reached peak injectate dispersion volumes of 955.84 mm³ and 791.94 mm³ at one (1) minute and zero (0) minutes, respectively. The EpiPen® Jr (Group P2) reached peak injectate dispersion volume (934.77 mm³) at zero (0) minutes. The spring force of the EpiPen® and EpiPen® Jr were the same (23.0 lbs).

Injectate uptake volumes for both EpiPen® and EpiPen® Jr were similar and relatively uniform in appearance. The difference in average uptake volumes at 15 minutes for EpiPen®—80% (Group P1) and 97% (Group P2) vs. EpiPen® Jr at 88% is not significant as it is within the variance noted within each trial.

5.6 Injections Administered Through Denim Patch Vs. Direct Skin Injections

There were no appreciable differences in either injectate dispersion or volume uptake for test (Anapen® 300, Twinject® 0.15 mL or Twinject® 0.30 mL) or control article (EpiPen® or EpiPen® Jr) injections with respect to whether the article was applied through denim or directly through the skin. It is noteworthy that data was analyzed with only two (2) animals per group. However, these data show that all auto-injector needles were able to successfully penetrate the denim, and injections through denim did not appear to affect any dispersion or uptake of study injectate.

### 5.7 Post-Injection Needle Lengths

All control and test article needle lengths approximated the needle lengths claimed on their respective labels. The variance of the three needle measurements for each article is within the lower bounds of the measurement resolution using this CT analysis method (the resolution bound being defined in relation to the voxel size of the CT dataset (0.015″×0.015″×0.04″). Since the length of the needles were along the z-axis of the CT scanner, the variability in needle length measurements between injectors can be seen to lie within the imaging resolution.

5.8 Post-Mortem Injection Site Skin/Fat Layer Measurement

The combined depth (mm) of the skin/fat layer directly on the injection site was measured post mortem. Measurements ranged from 1.65-3.57 mm. The average measure of this combined layer of the left thigh vs. the right thigh of all animals was similar (2.24 mm vs. 2.22 mm, respectively; averaging 2.23 mm). This data showed that auto-injections were given into muscle through a relatively uniform thickness of the skin/fat layer in all animals.

5.9 Conclusion Summary

The control article auto-injectors (EpiPen® and EpiPen® Jr) delivered injectate into the muscle tissue with greater efficiency than the test article auto-injectors (Anapen® 300, Twinject® 0.15 mL and Twinject® 0.30 mL). This efficiency was demonstrated by larger tissue dispersion volumes, more rapid peak dispersion volume and greater uptake of the injectate at the 15 minute post injection time point. Additionally, there was similarity in the pattern of injectate uptake between the EpiPen® and EpiPen® Jr and in the end injectate volume of uptake (80 and 97% vs. 88%, respectively). In contrast, while the Twinject® 0.30 mL auto-injector demonstrated a larger dispersion volume than either the Twinject® 0.15 mL or the Anapen® 300, none of the test articles displayed appreciable uptake of injectate, either in general or relative to one another, and remained essentially pooled in the tissue at the 15 minute time point as represented in FIG. 61.

No appreciable differences in injectate dispersion or uptake were noted when auto-injections were administered through denim vs. directly into the skin. The skin/fat layer at the injection sites were relatively uniform in thickness, and post-injection needle lengths were within acceptable variance of the labeled length.

### CONCLUSION

The test data discussed above, and particularly the results shown in FIGS. 60-74, show that the auto-injector apparatus and associated methods utilizing specific dimensions and parameters of use for the auto-injector as provided herein achieve increased effectiveness of the auto-injector device in delivering medicament into the patient's body, and in dispersion of the medicament from the initial injection site into the surrounding bodily tissues.

Without being bound by theory, these improved results may be attributable in part to one or more of the following factors taken alone or in combination:
a. The end surface area of the needle cover;
b. The force applied to the injection site by the end surface of the needle cover;
c. The time interval over which the end surface of the needle cover is held in place against the injection site after injection;
d. The volume of medicament to be delivered;
e. The size of the internal passage through the needle;
f. The injection depth;
g. The spring force applied to the plunger to expel the medicament;
h. The axial oscillatory motion of the needle within the patient's body tissue during the injection process;
i. The time interval required for injection of the medicament upon actuation of the device; and
j. The anti-kickback design of the auto-injector.

Rapid delivery of the bolus of medicament into the patient's body as a result of relatively large bore needle passages and high spring forces may result in increased tissue disruption thus providing channels within the tissue for subsequent uptake of the medicament into the surrounding tissue.

Maintenance of significant pressure on a relatively large surface area surrounding the injection site for a period of time after injection may aid in forcing the bolus of injected medicament to be taken up by the surrounding tissue. The surface area of contact surrounding the injection site for the EpiPen® and EpiPen® Jr. devices in the test was about 0.24 square inch, as contrasted to about 0.06 square inch and 0.08 square inch for the Twinject® and the Anapen® devices, respectively.

The test data suggest that the apparatus and methods of the present invention result in a larger average initial tissue dispersion volume of the medicament, which may be described as the medicament reaching a peak dispersion volume within the user's body of at least about 800 mm³, and more preferably at least about 900 mm³.

The test data suggest that the apparatus and methods of the present invention result in more rapid average peak dispersion volume of the medicament, which may be described as the medicament reaching a peak dispersion volume within the user's body within no more than about 2 minutes, and more preferably within no more than about 1 minute.

The test data suggest that the apparatus and methods of the present invention result in greater uptake of the injectate or medicament from the site of the injection 15 minutes post-injection, which may be described as achieving an uptake of the medicament from a peak dispersion volume into surrounding tissue of at least about 70% within 15 minutes post-injection, and more preferably at least about 80% within 15 minutes post-injection.

Thus, although there have been described particular embodiments of the present invention of a new and useful High Efficiency Auto-Injector, it is not intended that such references be construed as limitations upon the scope of this invention except as set forth in the following claims.

What is claimed is:

1. An auto-injector, comprising:
a housing;
a cartridge disposed in the housing and containing a medicament, the medicament rearwardly confined by a plunger, the cartridge including a needle to dispense the medicament there through, the needle having an inside diameter of at least 0.0115 inch;
an actuation assembly having a stored energy source capable of being released to drive the plunger within the cartridge to dispense the medicament through the needle, the energy source delivering a dynamic force of at least about 20 pounds to the plunger as the plunger begins moving relative to the cartridge; and
a needle cover at least partially received in the housing, the needle cover having an enclosed end surface having an end opening in the enclosed end surface to permit the needle to pass through the end opening during a medicament dispensing operation, the enclosed end surface having a flat planar annular portion surrounding the end opening and arranged to be placed on an injection surface of a user of the auto-injector to transmit an activation force to the actuation assembly when the auto-injector is pressed against the injection surface, the flat planar annular portion of the enclosed end surface having an area of at least 0.20 square inches.

2. The auto-injector of claim 1, wherein:
the needle inside diameter is at least 0.0125 inch.

3. The auto-injector of claim 1, wherein:
the needle inside diameter is at least 0.0155 inch.

4. The auto-injector of claim 1, wherein:
the needle inside diameter is no greater than 0.0345 inch.

5. The auto-injector of claim 1, wherein:
the energy source includes a coil compression spring having a static spring force of at least about 27 pounds prior to actuation.

6. The auto-injector of claim 1, wherein:
the energy source includes a coil compression spring having a static spring force of at least about 30 pounds prior to actuation.

7. The auto-injector of claim 1, wherein:
the dynamic force delivered by the energy source is at least about 22 pounds.

8. The auto-injector of claim 1, wherein:
the area of the flat planar annular portion of the enclosed end surface is at least 0.24 square inches.

9. The auto-injector of claim 1, wherein:
the needle inside diameter and the dynamic force delivered by the energy source are such that a medicament volume of at least 0.15 mL is dispensed through the needle into the user within no more than about 0.4 sec.

10. The auto-injector of claim 1, wherein:
the needle inside diameter and the dynamic force delivered by the energy source are such that a medicament volume of at least 0.30 mL is dispensed through the needle into the user within no more than about 0.4 sec.

11. The auto-injector of claim 1, wherein:
the plunger is movable within the cartridge by a distance sufficient to dispense at least 0.15 mL of medicament through the needle.

12. The auto-injector of claim 1, wherein:
the plunger is movable within the cartridge by a distance sufficient to dispense at least 0.30 mL of medicament through the needle.

13. The auto-injector of claim 1, wherein:
the needle is extendable through the needle cover to an injection depth in a range of from about 0.4 to 1.25 inch.

14. The auto-injector of claim 1, further comprising:
a collapsible resilient sheath disposed within the housing about the needle, the sheath arranged so that upon actuation of the actuation assembly the energy source must force the needle to pierce the sheath and to collapse the sheath within the housing.

15. The auto-injector of claim 14, wherein:
the energy source includes a spring; and
the spring and the collapsible resilient sheath are components of a dynamic spring, mass and dampener system of the auto-injector, with the spring and the collapsible resilient sheath contributing to an axial oscillatory motion of the needle immediately after the needle is extended to its maximum injection depth upon actuation of the actuation assembly.

16. A method of automatically injecting a medicament into a user, comprising:
(a) providing an auto-injector apparatus, including:
a housing;
a cartridge contained in the housing, the cartridge containing at least about 0.15 mL of medicament and including a plunger engaging the medicament and a needle connected to the cartridge;
an actuating assembly operably associated with the cartridge and the plunger; and
a needle guard operably associated with the actuating assembly;
(b) placing a flat planar end surface of the needle guard against an injection site of the user, the end surface having a surface area of at least about 0.20 square inches;
(c) pressing the end surface of the needle guard against the injection site with a force of at least about 2 pounds and thereby actuating the actuating assembly of the auto-injector apparatus so that:
(c)(1) the needle extends from the apparatus into the user, the needle having a needle bore diameter of at least 0.0115 inch; and
(c)(2) a force of at least about 20 pounds is applied by the plunger to the medicament so that at least about 0.15 mL of the medicament is expelled through the needle into the user within no more than about 0.5 second;
(d) after step (c), holding the end surface against the injection site for at least about 3 seconds; and
(e) after step (d), removing the end surface from contact with the injection site and automatically extending the end surface to cover the needle.

17. The method of claim 16, wherein:
in step (c)(1) the needle bore diameter is at least 0.0125 inch.

18. The method of claim 16, wherein:
in step (c)(1) the needle bore diameter is at least 0.0155 inch.

19. The method of claim 16, wherein:
in step (c)(1) the needle bore diameter is no greater than 0.0345 inch.

20. The method of claim 16, wherein:
in step (a) the actuating assembly includes a coil compression spring exerting a static spring force of at least about 27 pounds upon the cartridge prior to step (c).

21. The method of claim 20, wherein:
in step (a) the static spring force is at least about 30 pounds.

22. The method of claim 16, wherein:
in step (c)(2) the force applied to the medicament by the plunger is at least about 22 pounds.

23. The method of claim 16, wherein:
in step (b) the surface area of the flat planar end surface pressed against the injection site is at least about 0.24 square inches.

24. The method of claim 16, wherein:
in step (c)(2) at least 0.3 mL of medicament is expelled through the needle into the user within no more than about 0.4 sec.

25. The method of claim 16, wherein:
in step (c)(1) the needle extends to an injection depth in a range of from about 0.4 to 1.25 inch.

26. The method of claim 16, wherein:
in step (d) the end surface is held against the injection site for at least about 5 seconds.

27. The method of claim 16, wherein:
in step (d) the end surface is held against the injection site for at least about 10 seconds.

28. The method of claim 16, wherein:
the medicament reaches a peak dispersion volume within the user's body of at least about 800 mm³.

29. The method of claim 28, wherein:
the medicament reaches the peak dispersion volume within the user's body within no more than about 2 minutes.

30. The method of claim 28, wherein:
an uptake of the medicament from the peak dispersion volume into surrounding tissue of at least about 70% is achieved within 15 minutes post-injection.

31. The method of claim 16, wherein:
the medicament reaches a peak dispersion volume within the user's body of at least about 900 mm³.

32. The method of claim 16, wherein:
the medicament reaches a peak dispersion volume within the user's body within no more than about 1 minute.

33. The method of claim 16, wherein:
an uptake of the medicament from a peak dispersion volume into surrounding tissue of at least about 80% is achieved within 15 minutes post-injection.

34. The method of claim 16, wherein:
in step (a), the auto-injector apparatus includes a collapsible resilient sheath disposed within the housing about the needle, and the actuating assembly includes a spring power source;
in step (c), the needle pierces the sheath and the sheath collapses while being retained within the housing; and
in step (c), after the needle extends into the user the needle exhibits an axial oscillatory motion during at least part of the time the medicament is being expelled into the user.

35. The method of claim 16, wherein:
during step (d), the flat planar end surface of the needle guard compresses the user's body tissues around the needle to aid in sealing a puncture passage in the patient's body tissues to reduce flow back of injected medicament out of the puncture passage.
